(19) United States
(12) Patent Application Publication
(10) Pub. No.: US 2019/0389865 A1
(43) Pub. Date: Dec. 26, 2019

(54) SUBSTITUTED PYRROLO[1,2-a]TRIAZINES AND RELATED COMPOUNDS AND THEIR USE IN THE TREATMENT OF MEDICAL DISORDERS

(71) Applicant: Lysosomal Therapeutics Inc., Cambridge, MA (US)

(72) Inventors: Renato T. Skerlj, West Newton, MA (US); Elyse Marie Josee Bourque, L'etang-Du-Nord (CA); Peter T. Lansbury, Brookline, MA (US)

(21) Appl. No.: 16/097,902

(22) PCT Filed: May 5, 2017
(86) PCT No.: PCT/US2017/031187

§ 371 Date: Oct. 31, 2018

Related U.S. Application Data
(60) Provisional application No. 62/332,144, filed on May 5, 2016.

(51) Int. Cl. C07D 487/04; C07D 239/74

(52) U.S. Cl. CPC C07D 487/04; C07D 239/74

(57) ABSTRACT

The invention provides substituted pyrrolo[1,2-α]triazine compounds, related compounds, compositions containing such compounds, medical kits, and methods for using such compounds and compositions to treat medical disorders, e.g., Gaucher disease, Parkinson's disease, Lewy body disease, dementia, or multiple system atrophy, in a patient. Exemplary substituted pyrrolo[1,2-a]triazines compounds described herein include substituted 2,4-dimethyl-pyrrolo[1,2-α] [1,3,5]triazine-8-carboxamide compounds and variants thereof.

### CROSS-REFERENCE TO RELATED APPLICATIONS

This application is the national stage of International (PCT) Patent Application Serial No. PCT/US2017/031187, filed May 5, 2017, which claims the benefit of and priority to U.S. Provisional Patent Application Ser. No. 62/332,144, filed May 5, 2016, the contents of each of_which are hereby incorporated by reference.

### FIELD OF THE INVENTION

The invention provides substituted pyrrolo[1,2-a]triazine compounds, related compounds, compositions containing such compounds, medical kits, and methods for using such compounds and compositions to treat medical disorders in a patient.

### BACKGROUND

Gaucher disease is a genetic disorder associated with a deficiency of the lysosomal enzyme, glucocerebrosidase. Gaucher disease has been reported to have an incidence of approximately 1 in 20,000 live births in the general population, and it is a common lysosomal storage disorder. Current treatments for patients suffering from this disease include enzyme replacement therapy, which tends to be expensive, analgesics for bone pain relief, and medical procedures such as blood and platelet transfusions, splenectomy, and joint replacement for patients who experience bone erosion. However, new treatment options are needed having improved efficacy across a broader range of patients and/or reduced adverse side effects.

Mutations in the gene encoding glucocerebrosidase are also a risk factor for Parkinson's disease and diffuse Lewy Body Disease. Parkinson's disease is a degenerative disorder of the central nervous system associated with death of dopamine-containing cells in a region of the midbrain. Parkinson's disease afflicts millions of people, and the incidence of the disease increases with age. Treatment of Parkinson's disease frequently involves use of levodopa and dopamine agonists. However, these drugs can produce significant side effects such as hallucinations, insomnia, nausea, and constipation. Further, patients often develop tolerance to these drugs such that the drugs become ineffective at treating the symptoms of the disease, while sometimes also producing a movement disorder side effect called dyskinesia. Diffuse Lewy Body disease is a dementia that is sometimes confused with Alzheimer's disease.

Accordingly, the need exists for new therapeutic agents for treating Gaucher disease, Parkinson's disease, and related medical disorders. The present invention addresses this need and provides other related advantages.

### SUMMARY

The invention provides substituted pyrrolo[1,2-a]triazine compounds, related compounds, compositions containing such compounds, medical kits, and methods for using such compounds and compositions to treat medical disorders, e.g., Gaucher disease, Parkinson's disease, Lewy body disease, dementia, multiple system atrophy, epilepsy, bipolar disorder, schizophrenia, an anxiety disorder, major depression, polycystic kidney disease, type 2 diabetes, open angle glaucoma, multiple sclerosis, endometriosis, and multiple myeloma, in a patient. Various aspects and embodiments of the invention are described in further detail below.

Accordingly, one aspect of the invention provides a family of substituted pyrrolo[1,2-a]triazine compounds embraced by Formula I that may be used in the methods, compositions, and kits described herein, wherein Formula I is represented by:

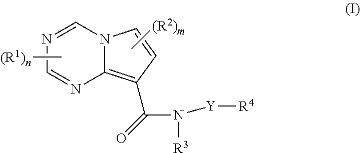

or a pharmaceutically acceptable salt thereof, wherein the variables are as defined in the detailed description.

Another aspect of the invention provides a family of substituted substituted quinazolinyl compounds embraced by Formula II that may be used in the methods, compositions, and kits described herein, wherein Formula II is represented by:

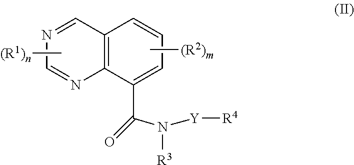

or a pharmaceutically acceptable salt thereof, wherein the variables are as defined in the detailed description.

Another aspect of the invention provides a pharmaceutical composition, comprising a pharmaceutically acceptable carrier and a substituted pyrrolo[1,2-a]triazine compound or related compound described herein, such as a compound of Formula I.

Another aspect of the invention provides a method of treating a disorder, e.g., Gaucher disease, Parkinson's disease, Lewy body disease, dementia, multiple system atrophy, epilepsy, bipolar disorder, schizophrenia, an anxiety disorder, major depression, polycystic kidney disease, type 2 diabetes, open angle glaucoma, multiple sclerosis, endometriosis, and multiple myeloma, in a patient. The method comprises administering to a patient in need thereof a therapeutically effective amount of a substituted pyrrolo[1,2-a]triazine compound or related compound described herein, such as a compound of Formula I, to treat the disorder, e.g., Gaucher disease, Parkinson's disease, Lewy body disease, dementia, multiple system atrophy, epilepsy, bipolar disorder, schizophrenia, an anxiety disorder, major depression, polycystic kidney disease, type 2 diabetes, open angle glaucoma, multiple sclerosis, endometriosis, or multiple myeloma.

### DETAILED DESCRIPTION

The invention provides substituted pyrrolo[1,2-a]triazine compounds, related compounds compositions containing such compounds, medical kits, and methods for using such compounds and compositions to treat medical disorders in a patient. The practice of the present invention employs, unless otherwise indicated, conventional techniques of organic chemistry, pharmacology, cell biology, and biochemistry. Such techniques are explained in the literature, such as in “Comprehensive Organic Synthesis” (B. M. Trost & I. Fleming, eds., 1991-1992); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety. Various aspects of the invention are set forth below in sections; however, aspects of the invention described in one particular section are not to be limited to any particular section.

### I. Definitions

To facilitate an understanding of the present invention, a number of terms and phrases are defined below.

The terms “a” and “an” as used herein mean “one or more” and include the plural unless the context is inappropriate.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. An exemplary alkylene group is —CH₂CH₂—.

The term “haloalkyl” refers to an alkyl group that is substituted with at least one halogen. For example, —CH₂F, —CHF₂, —CF₃, —CH₂CF₃, —CF₂CF₃, and the like.

The term “haloalkylene” refers to a diradical of a haloalkyl group. Exemplary haloalkylene groups are —CH₂CF₂— and —C(H)(CF₃)CH₂—.

The term “heteroalkyl” as used herein refers to an “alkyl” group in which at least one carbon atom has been replaced with a heteroatom (e.g., an O, N, or S atom). The heteroalkyl may be, for example, an —O—C₁-C₁₀alkyl group, an —C₁-C₆alkylene-O—C₁-C₆alkyl group, or a C₁-C₆ alkylene-OH group. In certain embodiments, the “heteroalkyl” may be 2-8 membered heteroalkyl, indicating that the heteroalkyl contains from 2 to 8 atoms selected from the group consisting of carbon, oxygen, nitrogen, and sulfur. In yet other embodiments, the heteroalkyl may be a 2-6 membered, 4-8 membered, or a 5-8 membered heteroalkyl group (which may contain for example 1 or 2 heteroatoms selected from the group oxygen and nitrogen). One type of heteroalkyl group is an “alkoxyl” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂alkenyl, C₂-C₁₀alkenyl, and C₂-C₆alkenyl, respectively. Exemplary alkenyl groups include vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, and the like.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂alkynyl, C₂-C₁₀alkynyl, and C₂-C₆alkynyl, respectively. Exemplary alkynyl groups include ethynyl, prop-1-yn-1-yl, and but-1-yn-1-yl.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈cycloalkyl,” derived from a cycloalkane. Unless specified otherwise, the cycloalkyl group is optionally substituted by 1 or 2 C₁-C₆ alkyl groups. In certain embodiments, the cycloalkyl group is unsubstituted. Exemplary cycloalkyl groups include, but are not limited to, cyclohexanes, cyclopentanes, cyclobutanes and cyclopropanes.

The term “cycloalkylene” refers to a diradical of an cycloalkyl group. An exemplary cycloalkylene group is

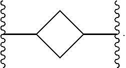

The term “cycloalkenyl” as used herein refers to a monovalent unsaturated cyclic, bicyclic, or bridged cyclic (e.g., adamantyl) hydrocarbon group of 3-12, 3-8, 4-8, or 4-6 carbons containing one carbon-carbon double bond, referred to herein, e.g., as “C₄₋₈cycloalkenyl,” derived from a cycloalkane. Exemplary cycloalkenyl groups include, but are not limited to, cyclohexenes, cyclopentenes, and cyclobutenes. Unless specified otherwise, cycloalkenyl groups are optionally substituted at one or more ring positions with, for example, alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkenyl group is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The term “aralkyl” refers to an alkyl group substituted with an aryl group.

The term “bicyclic carbocyclyl that is partially unsaturated” refers to a bicyclic carbocyclic group containing at least one double bond between ring atoms and at least one ring in the bicyclic carbocyclic group is not aromatic. Representative examples of a bicyclic carbocyclyl that is partially unsaturated include, for example:

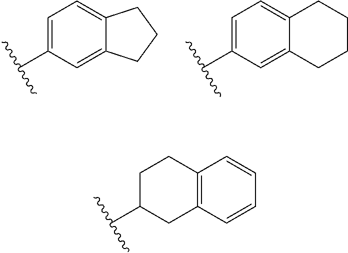 [SMILES: *c1ccc2c(c1)CCC2 ; *c1ccc2c(c1)CCCC2 ; *C1CCc2ccccc2C1]

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using Cₓ-Cₓ nomenclature where x is an integer specifying the number of ring atoms. For example, a C₃-C₇heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position. One example of a C₃heterocyclyl is aziridinyl. Heterocycles may also be mono-, bi-, or other multi-cyclic ring systems. A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isooxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. Unless specified otherwise, the heterocyclic ring is optionally substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, oxo, phosphate, phosphonato, phosphinato, sulfate, sulfide, sulfonamido, sulfonyl and thiocarbonyl. In certain embodiments, the heterocyclyl group is not substituted, i.e., it is unsubstituted.

The term “bicyclic heterocyclyl” refers to a heterocyclyl group that contains two rings that are fused together. Representative examples of a bicyclic heterocyclyl include, for example:

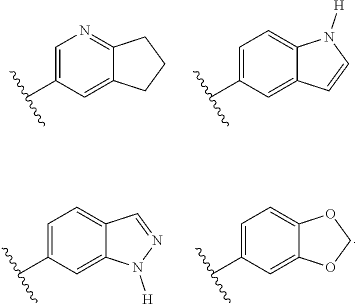 [SMILES: *c1cnc2c(c1)CCC2 ; *c1ccc2[nH]ccc2c1 ; *c1ccc2cn[nH]c2c1 ; *c1ccc2c(c1)OCO2]

In certain embodiments, the bicyclic heterocyclyl is an carbocyclic ring fused to partially unsaturated heterocyclic ring, that together form a bicyclic ring structure having 8-10 ring atoms (e.g., where there are 1, 2, 3, or 4 heteroatoms selected from the group consisting of nitrogen, oxygen, and sulfur).

The term “heterocycloalkyl” is art-recognized and refers to a saturated heterocyclyl group as defined above. In certain embodiments, the “heterocycloalkyl” is a 3- to 10-membered ring structures, alternatively a 3- to 7-membered rings, whose ring structures include one to four heteroatoms, such as nitrogen, oxygen, and sulfur.

The term “heterocycloalkylene” refers to a diradical of a heterocycloalkyl group. An exemplary heterocycloalkylene group is

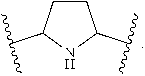

The heterocycloalkylene may contain, for example, 3-6 ring atom (i.e., a 3-6 membered heterocycloalkylene). In certain embodiments, the heterocycloalkylene is a 3-6 membered heterocycloalkylene containing 1, 2, or 3 three heteroatoms selected from the group consisting of oxygen, nitrogen, and sulfur.

The term “heteroaryl” is art-recognized and refers to aromatic groups that include at least one ring heteroatom. In certain instances, a heteroaryl group contains 1, 2, 3, or 4 ring heteroatoms. Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaryl ring is substituted at one or more ring positions with halogen, alkyl, hydroxyl, or alkoxyl. In certain other embodiments, the heteroaryl ring is not substituted, i.e., it is unsubstituted. In certain embodiments, the heteroaryl group is a 5- to 10-membered ring structure, alternatively a 5- to 6-membered ring structure, whose ring structure includes 1, 2, 3, or 4 heteroatoms, such as nitrogen, oxygen, and sulfur.

The term “heteroaralkyl” refers to an alkyl group substituted with a heteroaryl group.

The terms “amine” and “amino” are art-recognized and refer to both unsubstituted and substituted amines, e.g., a moiety represented by the general formula —N(R⁵⁰)(R⁵¹), wherein R⁵⁰ and R⁵¹ each independently represent hydrogen, alkyl, cycloalkyl, heterocyclyl, alkenyl, aryl, aralkyl, or —(CH₂)ₘ—R⁶¹; or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In certain embodiments, R⁵⁰ and R⁵¹ each independently represent hydrogen, alkyl, alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R₆₁, where m and R₆₁ are described above.

The term “carbamate” as used herein refers to a radical of the form —R_{g}OC(O)N(Rₕ)—, —R_{g}OC(O)N(Rₕ)Rᵢ—, or —OC(O)NRₕRᵢ, wherein R_{g}, Rₕ and Rᵢ are each independently alkoxy, aryloxy, alkyl, alkenyl, alkynyl, amide, amino, aryl, arylalkyl, carboxy, cyano, cycloalkyl, ester, ether, formyl, halogen, haloalkyl, heteroaryl, heterocyclyl, hydroxyl, ketone, nitro, sulfide, sulfonyl, or sulfonamide. Exemplary carbamates include arylcarbamates and heteroaryl carbamates, e.g., wherein at least one of R_{g}, Rₕ and Rᵢ are independently aryl or heteroaryl, such as phenyl and pyridinyl.

The term “carbonyl” as used herein refers to the radical —C(O)—.

The term “carboxamido” as used herein refers to the radical —C(O)NRR′, where R and R′ may be the same or different. R and R′ may be independently alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The term “amide” or “amido” as used herein refers to a radical of the form —RₐC(O)N(R_{b})—, —RₐC(O)N(R_{b})R_{c}—, —C(O)NR_{b}R_{c}, or —C(O)NH₂, wherein Rₐ, R_{b} and R_{c} are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro. The amide can be attached to another group through the carbon, the nitrogen, R_{b}, R_{c}, or Rₐ. The amide also may be cyclic, for example R_{b} and R_{c}, Rₐ and R_{b}, or Rₐ and R_{c} may be joined to form a 3- to 12-membered ring, such as a 3- to 10-membered ring or a 5- to 6-membered ring.

The term “amidino” as used herein refers to a radical of the form —C(═NR)NR′R″ where R, R′, and R″ are each independently alkyl, alkenyl, alkynyl, amide, aryl, arylalkyl, cyano, cycloalkyl, haloalkyl, heteroaryl, heterocyclyl, hydroxyl, ketone, or nitro.

The term “alkanoyl” as used herein refers to a radical —O—CO-alkyl.

The term “oxo” is art-recognized and refers to a “═O” substituent. For example, a cyclopentane susbsituted with an oxo group is cyclopentanone.

The term “sulfonamide” or “sulfonamido” as used herein refers to a radical having the structure —N(Rᵣ)—S(O)₂—Rₛ— or —S(O)₂—N(Rᵣ)Rₛ, where Rᵣ, and Rₛ can be, for example, hydrogen, alkyl, aryl, cycloalkyl, and heterocyclyl. Exemplary sulfonamides include alkylsulfonamides (e.g., where Rₛ is alkyl), arylsulfonamides (e.g., where Rₛ is aryl), cycloalkyl sulfonamides (e.g., where Rₛ is cycloalkyl), and heterocyclyl sulfonamides (e.g., where Rₛ is heterocyclyl), etc.

The term “sulfonyl” as used herein refers to a radical having the structure RᵤSO₂—, where Rᵤ can be alkyl, aryl, cycloalkyl, and heterocyclyl, e.g., alkylsulfonyl. The term “alkylsulfonyl” as used herein refers to an alkyl group attached to a sulfonyl group.

The symbol “ [symbol] ” indicates a point of attachment.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Further, enantiomers can be separated using supercritical fluid chromatographic (SFC) techniques described in the literature. Still further, stereoisomers can be obtained from stereomerically-pure intermediates, reagents, and catalysts by well-known asymmetric synthetic methods.

Geometric isomers can also exist in the compounds of the present invention. The symbol [symbol] denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E′” configuration wherein the terms “Z” and “E′” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in, e.g., the Examples herein by substituting an isotopically labeled reagent for a non-isotopically labeled reagent.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Abbreviations as used herein may include O-(7-azabenzotriazol-1-yl)-N,N,N′,N′-tetramethyluronium hexafluorophosphate (HATU); diisopropylethylamine (DIPEA); dimethylformamide (DMF); methylene chloride (DCM); tert-butoxycarbonyl (Boc); tetrahydrofuran (THF); trifluoroacetic acid (TFA); N-methylmorpholine (NMM); triethylamine (TEA); Boc anhydride ((Boc)₂O); dimethylsulfoxide (DMSO); diisopropylethylamine (DIEA); N,N-Dimethylpyridin-4-amine (DMAP); flash column chromatography (FCC); and supercritical fluid chromatography (SFC).

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. Substituted Pyrrolo[1,2-a]triazine Compounds & Related Compounds

One aspect of the invention provides substituted pyrrolo[1,2-a]triazine compounds. The substituted pyrrolo[1,2-a]triazine compounds are contemplated to be useful in the methods, compositions, and kits described herein. In certain embodiments, the substituted pyrrolo[1,2-a]triazine compound is a compound embraced by Formula I:

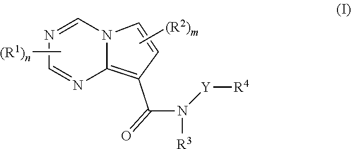

or a pharmaceutically acceptable salt thereof, wherein:

R¹ and R² each represent independently for each occurrence hydrogen, C₁-C₆ alkyl, halogen, C₁-C₆ haloalkyl, C₃-C₆ cycloalkyl, C₁-C₆ alkoxy, —(C₁-C₄ alkylene)-O—(C₁-C₆ alkyl), —(C₁-C₄ alkylene)-O—(C₃-C₆ cycloalkyl), 3-6 membered heterocyclyl, 6-membered aryl, cyano, or —N(R⁵)₂;

R³ is hydrogen, C₁-C₆ alkyl, or C₃-C₆ cycloalkyl;

R⁴ is one of the following:
C₃-C₈ cycloalkyl, 3-8 membered heterocycloalkyl, 9-13 membered spiroheterocycloalkyl, —(C₂-C₆ alkylene)-O-phenyl, phenyl, heteroaryl, a partially unsaturated 9-10 membered bicyclic carbocyclyl, or a partially unsaturated 8-10 membered bicyclic heterocyclyl; each of which is optionally substituted by 1, 2, or 3 substituents independently selected from the group consisting of C₁-C₈ alkyl, halogen, C₁-C₆ haloalkyl, C₃-C₆ cycloalkyl, —(C₁-C₆ alkylene,)—(C₃-C₆ cycloalkyl), hydroxyl, C₁-C₆ alkoxy, —O—(C₃-C₆ cycloalkyl), C₂-C₄ alkynyl, —(C₂-C₄ alkynyl)-C₁-C₆ alkoxy, aryl, —O-aryl, heteroaryl, saturated 3-8 membered heterocyclyl, amino, and —CO₂R⁵; or
C₁-C₆ alkyl;

R⁵ represents independently for each occurrence hydrogen, C₁-C₆ alkyl, or C₃-C₆ cycloalkyl;

Y is a bond, —C(O)—, C₁-C₆ alkylene, C₁-C₆ haloalkylene, or C₃-C₆ cycloalkylene; and

n and m are independently 1 or 2.

Definitions of the variables in Formula I above encompass multiple chemical groups. The application contemplates embodiments where, for example, i) the definition of a variable is a single chemical group selected from those chemical groups set forth above, ii) the definition is a collection of two or more of the chemical groups selected from those set forth above, and iii) the compound is defined by a combination of variables in which the variables are defined by (i) or (ii), e.g., such as where R¹ is C₁-C₆ alkyl, R² is hydrogen, and R⁴ is C₃-C₈ cycloalkyl or phenyl; each of which is optionally substituted by 1, 2, or 3 substituents independently selected from the group consisting of C₁-C₈ alkyl, halogen, C₁-C₆ haloalkyl, and C₃-C₆ cycloalkyl.

Accordingly, in certain embodiments, R¹ is C₁-C₆ alkyl. Accordingly, in certain embodiments, R¹ is C₁-C₃ alkyl.

In certain embodiments, R² is hydrogen. In certain embodiments, R² is C₁-C₃ alkyl.

In certain embodiments, R³ is hydrogen.

In certain embodiments, Y is a bond. In certain embodiments, Y is C₁-C₆ alkylene.

In certain embodiments, R⁴ is C₃-C₈ cycloalkyl optionally substituted by 1, 2, or 3 substituents independently selected from the group consisting of C₁-C₈ alkyl, halogen, C₁-C₆ haloalkyl, C₃-C₆ cycloalkyl, —(C₁-C₆ alkylene,)—(C₃-C₆ cycloalkyl), hydroxyl, C₁-C₆ alkoxy, C₂-C₄ alkynyl, —(C₂-C₄ alkynyl)-C₁-C₆ alkoxy, aryl, —O-aryl, heteroaryl, saturated 3-8 membered heterocyclyl, amino, and —CO₂R⁵. In certain embodiments, R⁴ is C₃-C₈ cycloalkyl optionally substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, and —(C₂-C₄ alkynyl)-C₁-C₆ alkoxy. In certain embodiments, R⁴ is C₄-C₆ cycloalkyl substituted by 1, 2, or 3 substituents independently selected from the group consisting of C₁-C₆ alkyl, C₁-C₆ haloalkyl, C₃-C₆ cycloalkyl, and C₁-C₆ alkoxy.

In certain embodiments, R⁴ is a partially unsaturated 9-10 membered bicyclic carbocyclyl optionally substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, —(C₂-C₄ alkynyl)-C₁-C₆ alkoxy, aryl, heteroaryl, and saturated 3-8 membered heterocyclyl. In certain embodiments, R⁴ is a partially unsaturated 9-10 membered bicyclic carbocyclyl optionally substituted by C₁-C₆ alkyl.

In certain embodiments, R⁴ is phenyl substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, —(C₂-C₄ alkynyl)-C₁-C₆ alkoxy, 5-membered heteroaryl, and saturated 3-8 membered heterocyclyl. In certain embodiments, R⁴ is phenyl substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, and —(C₂-C₄ alkynyl)-C₁-C₆ alkoxy. In certain embodiments, R⁴ is phenyl substituted by 1, 2, or 3 substituents independently selected from the group consisting of C₁-C₆ alkyl, halogen, C₁-C₆ haloalkyl, C₃-C₆ cycloalkyl, C₂-C₄ alkynyl, and —(C₂-C₄ alkynyl)-C₁-C₆ alkoxy. In certain embodiments, R⁴ is C₃-C₈ cycloalkyl or phenyl; each of which is optionally substituted by 1, 2, or 3 substituents independently selected from the group consisting of C₁-C₈ alkyl, halogen, C₁-C₆ haloalkyl, and C₃-C₆ cycloalkyl

In certain embodiments, n is 2. In certain embodiments, m is 1.

The description above describes multiple embodiments relating to compounds of Formula I. The patent application specifically contemplates all combinations of the embodiments.

In certain embodiments, the substituted pyrrolo[1,2-a]triazine compound is a compound embraced by Formula I-1:

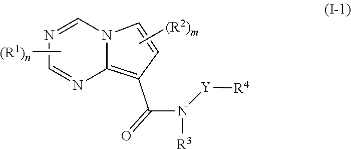

or a pharmaceutically acceptable salt thereof, wherein:

R¹ and R² each represent independently for each occurrence hydrogen, C₁-C₆ alkyl, halogen, C₁-C₆ haloalkyl, or C₃-C₆ cycloalkyl;

R³ is hydrogen, C₁-C₆ alkyl, or C₃-C₆ cycloalkyl;

R⁴ is C₃-C₈ cycloalkyl, 3-8 membered heterocycloalkyl, 9-13 membered spiroheterocycloalkyl, —(C₂-C₆ alkylene)-O-phenyl, phenyl, heteroaryl, a partially unsaturated 9-10 membered bicyclic carbocyclyl, or a partially unsaturated 8-10 membered bicyclic heterocyclyl; each of which is optionally substituted by 1, 2, or 3 substituents independently selected from the group consisting of C₁-C₈ alkyl, halogen, C₁-C₆ haloalkyl, C₃-C₆ cycloalkyl, —(C₁-C₆ alkylene,)—(C₃-C₆ cycloalkyl), hydroxyl, C₁-C₆ alkoxy, C₂-C₄ alkynyl, —(C₂-C₄ alkynyl)-C₁-C₆ alkoxy, aryl, —O-aryl, heteroaryl, saturated 3-8 membered heterocyclyl, amino, and —CO₂R⁵;

R⁵ represents independently for each occurrence hydrogen, C₁-C₆ alkyl, or C₃-C₆ cycloalkyl;

Y is a bond or C₁-C₆ alkylene; and

n and m are independently 1 or 2.

Definitions of the variables in Formula I-1 above encompass multiple chemical groups. The application contemplates embodiments where, for example, i) the definition of a variable is a single chemical group selected from those chemical groups set forth above, ii) the definition is a collection of two or more of the chemical groups selected from those set forth above, and iii) the compound is defined by a combination of variables in which the variables are defined by (i) or (ii), e.g., such as where R¹ is C₁-C₆ alkyl, R² is hydrogen, and R⁴ is C₃-C₈ cycloalkyl or phenyl; each of which is optionally substituted by 1, 2, or 3 substituents independently selected from the group consisting of C₁-C₈ alkyl, halogen, C₁-C₆ haloalkyl, and C₃-C₆ cycloalkyl.

Accordingly, in certain embodiments, R¹ is C₁-C₆ alkyl. Accordingly, in certain embodiments, R¹ is C₁-C₃ alkyl.

In certain embodiments, R² is hydrogen. In certain embodiments, R² is C₁-C₃ alkyl.

In certain embodiments, R³ is hydrogen.

In certain embodiments, Y is a bond. In certain embodiments, Y is C₁-C₆ alkylene.

In certain embodiments, R⁴ is C₃-C₈ cycloalkyl optionally substituted by 1, 2, or 3 substituents independently selected from the group consisting of C₁-C₈ alkyl, halogen, C₁-C₆ haloalkyl, C₃-C₆ cycloalkyl, —(C₁-C₆ alkylene,)—(C₃-C₆ cycloalkyl), hydroxyl, C₁-C₆ alkoxy, C₂-C₄ alkynyl, —(C₂-C₄ alkynyl)-C₁-C₆ alkoxy, aryl, —O-aryl, heteroaryl, saturated 3-8 membered heterocyclyl, amino, and —CO₂R⁵. In certain embodiments, R⁴ is C₃-C₈ cycloalkyl optionally substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, and —(C₂-C₄ alkynyl)-C₁-C₆ alkoxy. In certain embodiments, R⁴ is C₄-C₆ cycloalkyl substituted by 1, 2, or 3 substituents independently selected from the group consisting of C₁-C₆ alkyl, C₁-C₆ haloalkyl, C₃-C₆ cycloalkyl, and C₁-C₆ alkoxy.

In certain embodiments, R⁴ is a partially unsaturated 9-10 membered bicyclic carbocyclyl optionally substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, —(C₂-C₄ alkynyl)-C₁-C₆ alkoxy, aryl, heteroaryl, and saturated 3-8 membered heterocyclyl. In certain embodiments, R⁴ is a partially unsaturated 9-10 membered bicyclic carbocyclyl optionally substituted by C₁-C₆ alkyl.

In certain embodiments, R⁴ is phenyl substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, —(C₂-C₄ alkynyl)-C₁-C₆ alkoxy, 5-membered heteroaryl, and saturated 3-8 membered heterocyclyl. In certain embodiments, R⁴ is phenyl substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, and —(C₂-C₄ alkynyl)-C₁-C₆ alkoxy. In certain embodiments, R⁴ is phenyl substituted by 1, 2, or 3 substituents independently selected from the group consisting of C₁-C₆ alkyl, halogen, C₁-C₆ haloalkyl, C₃-C₆ cycloalkyl, C₂-C₄ alkynyl, and —(C₂-C₄ alkynyl)-C₁-C₆ alkoxy. In certain embodiments, R⁴ is C₃-C₈ cycloalkyl or phenyl; each of which is optionally substituted by 1, 2, or 3 substituents independently selected from the group consisting of C₁-C₈ alkyl, halogen, C₁-C₆ haloalkyl, and C₃-C₆ cycloalkyl

In certain embodiments, n is 2. In certain embodiments, m is 1.

The description above describes multiple embodiments relating to compounds of Formula I-1. The patent application specifically contemplates all combinations of the embodiments.

In certain embodiments, the compound is a compound of Formula I-A:

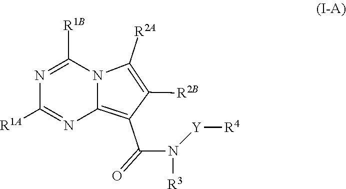

or a pharmaceutically acceptable salt thereof, wherein:

R^{1A}, R^{1B}, R^{2A}, and R^{2B} are independently hydrogen, C₁-C₃ alkyl, or cyclopropyl;

R³ is hydrogen;

R⁴ is C₃-C₈ cycloalkyl, 3-8 membered heterocycloalkyl, 9-13 membered spiroheterocycloalkyl, —(C₂-C₆ alkylene)-O-phenyl, phenyl, heteroaryl, a partially unsaturated 9-10 membered bicyclic carbocyclyl, or a partially unsaturated 8-10 membered bicyclic heterocyclyl; each of which is optionally substituted by 1, 2, or 3 substituents independently selected from the group consisting of C₁-C₈ alkyl, halogen, C₁-C₆ haloalkyl, C₃-C₆ cycloalkyl, —(C₁-C₆ alkylene,)—(C₃-C₆ cycloalkyl), hydroxyl, C₁-C₆ alkoxy, C₂-C₄ alkynyl, and —(C₂-C₄ alkynyl)-C₁-C₆ alkoxy; and

Y is a bond or C₁-C₆ alkylene.

Definitions of the variables in Formula I-A above encompass multiple chemical groups. The application contemplates embodiments where, for example, i) the definition of a variable is a single chemical group selected from those chemical groups set forth above, ii) the definition is a collection of two or more of the chemical groups selected from those set forth above, and iii) the compound is defined by a combination of variables in which the variables are defined by (i) or (ii), e.g., such as where R^{1A} and R^{1B} are C₁-C₃ alkyl, R^{2A} and R^{2B} are hydrogen, and R⁴ is C₃-C₈ cycloalkyl or phenyl; each of which is optionally substituted by 1, 2, or 3 substituents independently selected from the group consisting of C₁-C₈ alkyl, halogen, C₁-C₆ haloalkyl, and C₃-C₆ cycloalkyl.

Accordingly, in certain embodiments, R^{1A} and R^{1B} are methyl. In certain embodiments, R^{1A} and R^{1B} are C₁-C₃ alkyl.

In certain embodiments, R^{2A} and R^{2B} are hydrogen.

In certain embodiments, Y is a bond. In certain embodiments, Y is C₁-C₆ alkylene.

In certain embodiments, R⁴ is C₃-C₈ cycloalkyl optionally substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, and —(C₂-C₄ alkynyl)-C₁-C₆ alkoxy. In certain embodiments, R⁴ is C₃-C₈ cycloalkyl optionally substituted by 1, 2, or 3 substituents independently selected from the group consisting of C₁-C₆ alkyl, C₁-C₆ haloalkyl, C₃-C₆ cycloalkyl, C₁-C₆ alkoxy, and C₂-C₄ alkynyl. In certain embodiments, R⁴ is C₄-C₆ cycloalkyl substituted by 1, 2, or 3 substituents independently selected from the group consisting of C₁-C₆ alkyl, C₁-C₆ haloalkyl, C₃-C₆ cycloalkyl, and C₁-C₆ alkoxy. In certain embodiments, R⁴ is C₄-C₆ cycloalkyl optionally substituted by 1 or 2 substituents independently selected from the group consisting of C₁-C₆ alkyl, C₁-C₆ haloalkyl, C₃-C₆ cycloalkyl, and C₁-C₆ alkoxy.

In certain embodiments, R⁴ is a partially unsaturated 9-10 membered bicyclic carbocyclyl optionally substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, and —(C₂-C₄ alkynyl)-C₁-C₆ alkoxy. In certain embodiments, R⁴ is a partially unsaturated 9-10 membered bicyclic carbocyclyl optionally substituted by C₁-C₆ alkyl.

In certain embodiments, R⁴ is phenyl substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, and —(C₂-C₄ alkynyl)-C₁-C₆ alkoxy. In certain embodiments, R⁴ is phenyl substituted by 1, 2, or 3 substituents independently selected from the group consisting of C₁-C₆ alkyl, halogen, C₁-C₆ haloalkyl, C₃-C₆ cycloalkyl, C₁-C₆ alkoxy, and C₂-C₄ alkynyl. In certain embodiments, R⁴ is phenyl substituted by 1, 2, or 3 substituents independently selected from the group consisting of C₁-C₆ alkyl, halogen, C₁-C₆ haloalkyl, C₃-C₆ cycloalkyl, C₂-C₄ alkynyl, and —(C₂-C₄ alkynyl)-C₁-C₆ alkoxy. In certain embodiments, R⁴ is C₃-C₈ cycloalkyl or phenyl; each of which is optionally substituted by 1, 2, or 3 substituents independently selected from the group consisting of C₁-C₈ alkyl, halogen, C₁-C₆ haloalkyl, and C₃-C₆ cycloalkyl.

The description above describes multiple embodiments relating to compounds of Formula I-A. The patent application specifically contemplates all combinations of the embodiments.

In certain other embodiments, the compound is one of the compounds listed in Table 1 or 2 below or a pharmaceutically acceptable salt thereof.

TABLE 1

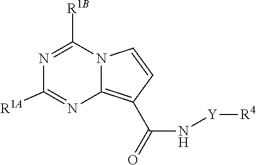

| Compound | | | | |
|---|---|---|---|---|
| No. | R^{1A} | R^{1B} | Y | R⁴ |
| I-1 | —CH₃ | —OCH₃ | a bond | 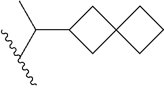 [SMILES: *C(C)C1CC2(CCC2)C1] |
| I-2 | —OCH₃ | —CH₃ | a bond | 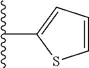 [SMILES: *c1cccs1] |
| I-3 | —CH₃ | —N(H)CH₃ | a bond | 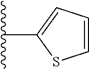 [SMILES: *c1cccs1] |
| I-4 | —N(H)CH₃ | —CH₃ | a bond | 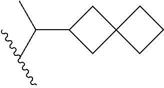 [SMILES: *C(C)C1CC2(CCC2)C1] |
| I-5 | —CH₃ | —CH₃ | a bond | 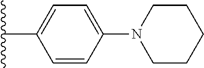 [SMILES: *c1ccc(N2CCCCC2)cc1] |
| I-6 | —CH₃ | —CH₃ | a bond | 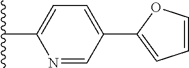 [SMILES: *c1ccc(-c2ccco2)cn1] |
| I-7 | —CH₃ | —CH₃ | a bond | 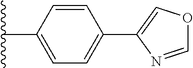 [SMILES: *c1ccc(-c2cocn2)cc1] |
| I-8 | —CH₃ | —CH₃ | a bond | 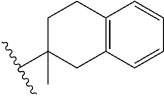 [SMILES: *C1(C)CCc2ccccc2C1] |
| I-9 | —N(H)CH₃ | —CH₃ | a bond | 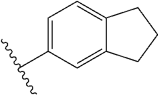 [SMILES: *c1ccc2c(c1)CCC2] |
| I-10 | —CH₃ | —CH₃ | a bond | 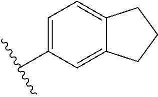 [SMILES: *c1ccc2c(c1)CCC2] |
| I-11 | —OCH₃ | —CH₃ | a bond | 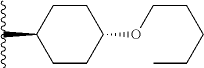 [SMILES: *[C@H]1CC[C@H](OCCCCC)CC1] |
| I-12 | —N(H)CH₃ | —CH₃ | a bond | 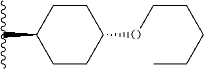 [SMILES: *[C@H]1CC[C@H](OCCCCC)CC1] |
| I-13 | —CH₃ | —CH₃ | a bond | 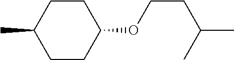 |
| I-14 | —CH₃ | —CH₃ | a bond | 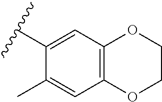 [SMILES: *c1cc2c(cc1C)OCCO2] |
| I-15 | —CH₃ | —CH₃ | C₁-C₄ alkylene | 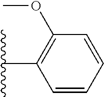 [SMILES: *c1ccccc1OC] |
| I-16 | —CH₃ | —CH₃ | C₁-C₄ alkylene | 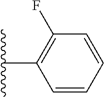 [SMILES: *c1ccccc1F] |
| I-17 | —CH₃ | —CH₃ | C₁-C₄ alkylene | 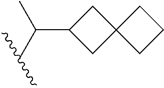 [SMILES: *C(C)C1CC2(CCC2)C1] |
| I-18 | —CH₃ | —CH₃ | C₁-C₄ alkylene | 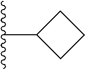 [SMILES: *C1CCC1] |
| I-19 | —CH₃ | —CH₃ | C₁-C₄ alkylene | 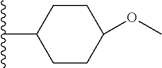 [SMILES: *C1CCC(OC)CC1] |

TABLE 2

| Com- | |
|---|---|
| pound | |
| No. | Compound Structure |
| II-1 | 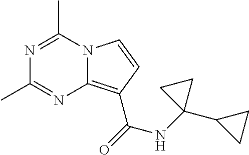 [SMILES: Cc1nc(C)n2ccc(C(=O)NC3(C4CC4)CC3)c2n1] |
| II-2 | 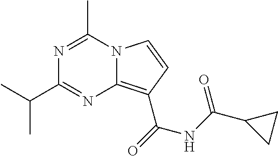 [SMILES: Cc1nc(C(C)C)nc2c(C(=O)NC(=O)C3CC3)ccn12] |
| II-3 | 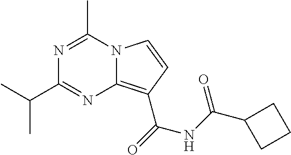 [SMILES: Cc1nc(C(C)C)nc2c(C(=O)NC(=O)C3CCC3)ccn12] |
| II-4 | 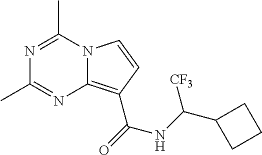 [SMILES: Cc1nc(C)n2ccc(C(=O)NC(C3CCC3)C(F)(F)F)c2n1] |
| II-5 | 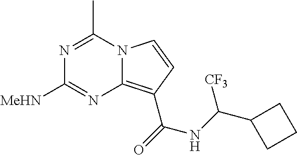 [SMILES: CNc1nc(C)n2ccc(C(=O)NC(C3CCC3)C(F)(F)F)c2n1] |
| II-6 | 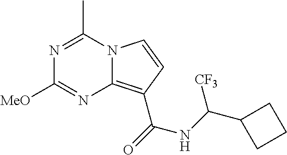 [SMILES: COc1nc(C)n2ccc(C(=O)NC(C3CCC3)C(F)(F)F)c2n1] |
| II-7 | 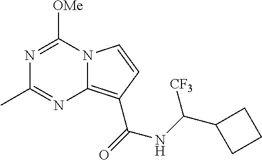 [SMILES: COc1nc(C)nc2c(C(=O)NC(C3CCC3)C(F)(F)F)ccn12] |
| II-8 | 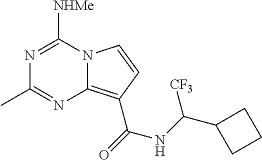 [SMILES: CNc1nc(C)nc2c(C(=O)NC(C3CCC3)C(F)(F)F)ccn12] |
| II-9 | 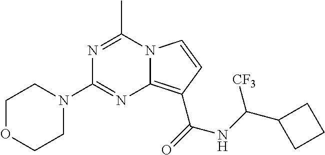 [SMILES: Cc1nc(N2CCOCC2)nc2c(C(=O)NC(C3CCC3)C(F)(F)F)ccn12] |
| II-10 | 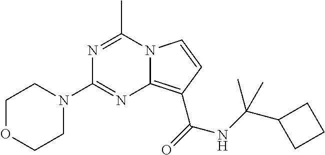 [SMILES: Cc1nc(N2CCOCC2)nc2c(C(=O)NC(C)(C)C3CCC3)ccn12] |
| II-11 | 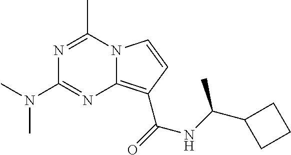 [SMILES: Cc1nc(N(C)C)nc2c(C(=O)N[C@@H](C)C3CCC3)ccn12] |
| II-12 | 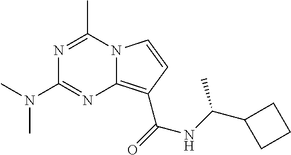 [SMILES: Cc1nc(N(C)C)nc2c(C(=O)N[C@H](C)C3CCC3)ccn12] |
| II-13 | 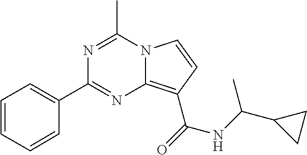 [SMILES: Cc1nc(-c2ccccc2)nc2c(C(=O)NC(C)C3CC3)ccn12] |
| II-14 | 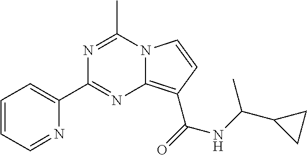 [SMILES: Cc1nc(-c2ccccn2)nc2c(C(=O)NC(C)C3CC3)ccn12] |
| II-15 | 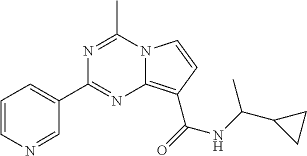 [SMILES: Cc1nc(-c2cccnc2)nc2c(C(=O)NC(C)C3CC3)ccn12] |
| II-16 | 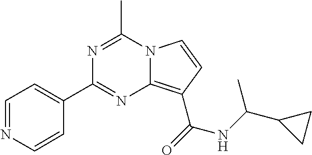 [SMILES: Cc1nc(-c2ccncc2)nc2c(C(=O)NC(C)C3CC3)ccn12] |
| II-17 | 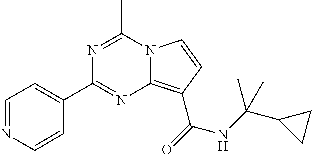 [SMILES: Cc1nc(-c2ccncc2)nc2c(C(=O)NC(C)(C)C3CC3)ccn12] |
| II-18 | 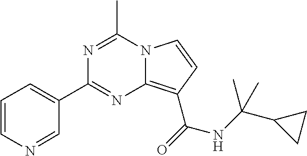 [SMILES: Cc1nc(-c2cccnc2)nc2c(C(=O)NC(C)(C)C3CC3)ccn12] |
| II-19 | 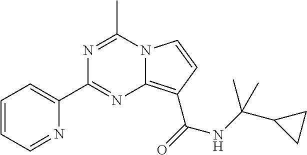 [SMILES: Cc1nc(-c2ccccn2)nc2c(C(=O)NC(C)(C)C3CC3)ccn12] |
| II-20 | 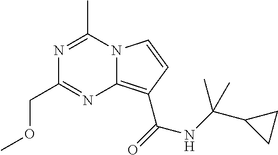 [SMILES: COCc1nc(C)n2ccc(C(=O)NC(C)(C)C3CC3)c2n1] |
| II-21 | 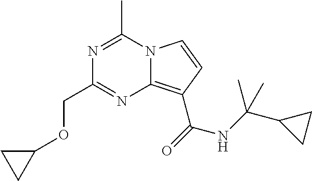 [SMILES: Cc1nc(COC2CC2)nc2c(C(=O)NC(C)(C)C3CC3)ccn12] |
| II-22 | 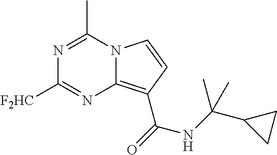 [SMILES: Cc1nc(C(F)F)nc2c(C(=O)NC(C)(C)C3CC3)ccn12] |
| II-23 | 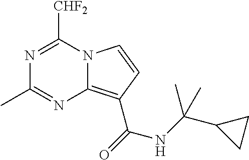 [SMILES: Cc1nc(C(F)F)n2ccc(C(=O)NC(C)(C)C3CC3)c2n1] |
| II-24 | 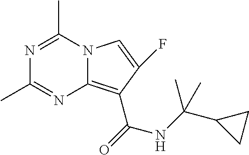 [SMILES: Cc1nc(C)n2cc(F)c(C(=O)NC(C)(C)C3CC3)c2n1] |
| II-25 | 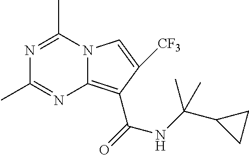 [SMILES: Cc1nc(C)n2cc(C(F)(F)F)c(C(=O)NC(C)(C)C3CC3)c2n1] |
| II-26 | 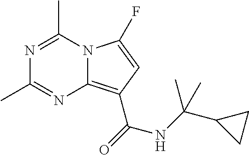 [SMILES: Cc1nc(C)n2c(F)cc(C(=O)NC(C)(C)C3CC3)c2n1] |
| II-27 | 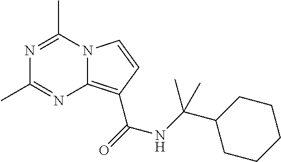 [SMILES: Cc1nc(C)n2ccc(C(=O)NC(C)(C)C3CCCCC3)c2n1] |
| II-28 | 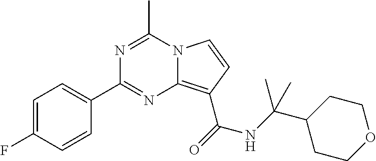 [SMILES: Cc1nc(-c2ccc(F)cc2)nc2c(C(=O)NC(C)(C)C3CCOCC3)ccn12] |
| II-29 | 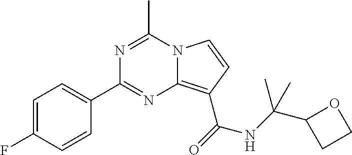 [SMILES: Cc1nc(-c2ccc(F)cc2)nc2c(C(=O)NC(C)(C)C3CCO3)ccn12] |

Another aspect of the invention provides substituted quinazolinyl compounds. The substituted quinazolinyl compounds are contemplated to be useful in the methods, compositions, and kits described herein. In certain embodiments, the substituted quinazolinyl compound is a compound embraced by Formula II:

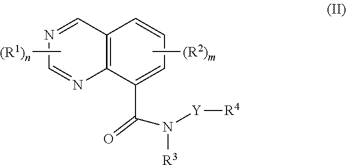

or a pharmaceutically acceptable salt thereof, wherein:

R¹ and R² each represent independently for each occurrence hydrogen, C₁-C₆ alkyl, halogen, C₁-C₆ haloalkyl, C₃-C₆ cycloalkyl, C₁-C₆ alkoxy, —(C₁-C₄ alkylene)-O—(C₁-C₆ alkyl), —(C₁-C₄ alkylene)-O—(C₃-C₆ cycloalkyl), 3-6 membered heterocyclyl, 6-membered aryl, cyano, or —N(R⁵)₂;

R³ is hydrogen, C₁-C₆ alkyl, or C₃-C₆ cycloalkyl;

R⁴ is one of the following:
C₃-C₈ cycloalkyl, 3-8 membered heterocycloalkyl, 9-13 membered spiroheterocycloalkyl, —(C₂-C₆ alkylene)-O-phenyl, phenyl, heteroaryl, a partially unsaturated 9-10 membered bicyclic carbocyclyl, or a partially unsaturated 8-10 membered bicyclic heterocyclyl; each of which is optionally substituted by 1, 2, or 3 substituents independently selected from the group consisting of C₁-C₈ alkyl, halogen, C₁-C₆ haloalkyl, C₃-C₆ cycloalkyl, —(C₁-C₆ alkylene,)—(C₃-C₆ cycloalkyl), hydroxyl, C₁-C₆ alkoxy, —O—(C₃-C₆ cycloalkyl), C₂-C₄ alkynyl, —(C₂-C₄ alkynyl)-C₁-C₆ alkoxy, aryl, —O-aryl, heteroaryl, saturated 3-8 membered heterocyclyl, amino, and —CO₂R⁵; or
C₁-C₆ alkyl;

R⁵ represents independently for each occurrence hydrogen, C₁-C₆ alkyl, or C₃-C₆ cycloalkyl;

Y is a bond, —C(O)—, C₁-C₆ alkylene, C₁-C₆ haloalkylene, or C₃-C₆ cycloalkylene; and

n and m are independently 1 or 2.

Definitions of the variables in Formula I above encompass multiple chemical groups. The application contemplates embodiments where, for example, i) the definition of a variable is a single chemical group selected from those chemical groups set forth above, ii) the definition is a collection of two or more of the chemical groups selected from those set forth above, and iii) the compound is defined by a combination of variables in which the variables are defined by (i) or (ii), e.g., such as where R¹ is C₁-C₆ alkyl, R² is hydrogen, and R⁴ is C₃-C₈ cycloalkyl or phenyl; each of which is optionally substituted by 1, 2, or 3 substituents independently selected from the group consisting of C₁-C₈ alkyl, halogen, C₁-C₆ haloalkyl, and C₃-C₆ cycloalkyl.

Accordingly, in certain embodiments, R¹ is C₁-C₆ alkyl. Accordingly, in certain embodiments, R¹ is C₁-C₃ alkyl.

In certain embodiments, R² is hydrogen. In certain embodiments, R² is C₁-C₃ alkyl.

In certain embodiments, R³ is hydrogen.

In certain embodiments, Y is a bond. In certain embodiments, Y is C₁-C₆ alkylene.

In certain embodiments, R⁴ is C₃-C₈ cycloalkyl optionally substituted by 1, 2, or 3 substituents independently selected from the group consisting of C₁-C₈ alkyl, halogen, C₁-C₆ haloalkyl, C₃-C₆ cycloalkyl, —(C₁-C₆ alkylene,)—(C₃-C₆ cycloalkyl), hydroxyl, C₁-C₆ alkoxy, C₂-C₄ alkynyl, —(C₂-C₄ alkynyl)-C₁-C₆ alkoxy, aryl, —O-aryl, heteroaryl, saturated 3-8 membered heterocyclyl, amino, and —CO₂R⁵. In certain embodiments, R⁴ is C₃-C₈ cycloalkyl optionally substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, and —(C₂-C₄ alkynyl)-C₁-C₆ alkoxy. In certain embodiments, R⁴ is C₄-C₆ cycloalkyl substituted by 1, 2, or 3 substituents independently selected from the group consisting of C₁-C₆ alkyl, C₁-C₆ haloalkyl, C₃-C₆ cycloalkyl, and C₁-C₆ alkoxy.

In certain embodiments, R⁴ is a partially unsaturated 9-10 membered bicyclic carbocyclyl optionally substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, —(C₂-C₄ alkynyl)-C₁-C₆ alkoxy, aryl, heteroaryl, and saturated 3-8 membered heterocyclyl. In certain embodiments, R⁴ is a partially unsaturated 9-10 membered bicyclic carbocyclyl optionally substituted by C₁-C₆ alkyl.

In certain embodiments, R⁴ is phenyl substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, —(C₂-C₄ alkynyl)-C₁-C₆ alkoxy, 5-membered heteroaryl, and saturated 3-8 membered heterocyclyl. In certain embodiments, R⁴ is phenyl substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, and —(C₂-C₄ alkynyl)-C₁-C₆ alkoxy. In certain embodiments, R⁴ is phenyl substituted by 1, 2, or 3 substituents independently selected from the group consisting of C₁-C₆ alkyl, halogen, C₁-C₆ haloalkyl, C₃-C₆ cycloalkyl, C₂-C₄ alkynyl, and —(C₂-C₄ alkynyl)-C₁-C₆ alkoxy. In certain embodiments, R⁴ is C₃-C₈ cycloalkyl or phenyl; each of which is optionally substituted by 1, 2, or 3 substituents independently selected from the group consisting of C₁-C₈ alkyl, halogen, C₁-C₆ haloalkyl, and C₃-C₆ cycloalkyl

In certain embodiments, n is 2. In certain embodiments, m is 1.

The description above describes multiple embodiments relating to compounds of Formula II. The patent application specifically contemplates all combinations of the embodiments.

In certain embodiments, the compound is a compound of Formula II-A:

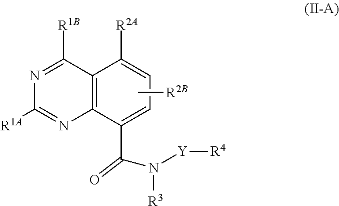

or a pharmaceutically acceptable salt thereof, wherein:

R^{1A}, R^{1B}, R^{2A}, and R^{2B} are independently hydrogen, C₁-C₃ alkyl, or cyclopropyl;

R³ is hydrogen;

R⁴ is C₃-C₈ cycloalkyl, 3-8 membered heterocycloalkyl, 9-13 membered spiroheterocycloalkyl, —(C₂-C₆ alkylene)-O-phenyl, phenyl, heteroaryl, a partially unsaturated 9-10 membered bicyclic carbocyclyl, or a partially unsaturated 8-10 membered bicyclic heterocyclyl; each of which is optionally substituted by 1, 2, or 3 substituents independently selected from the group consisting of C₁-C₈ alkyl, halogen, C₁-C₆ haloalkyl, C₃-C₆ cycloalkyl, —(C₁-C₆ alkylene,)—(C₃-C₆ cycloalkyl), hydroxyl, C₁-C₆ alkoxy, C₂-C₄ alkynyl, and —(C₂-C₄ alkynyl)-C₁-C₆ alkoxy; and

Y is a bond or C₁-C₆ alkylene.

Definitions of the variables in Formula II-A above encompass multiple chemical groups. The application contemplates embodiments where, for example, i) the definition of a variable is a single chemical group selected from those chemical groups set forth above, ii) the definition is a collection of two or more of the chemical groups selected from those set forth above, and iii) the compound is defined by a combination of variables in which the variables are defined by (i) or (ii), e.g., such as where R^{1A} and R^{1B} are C₁-C₃ alkyl, R^{2A} and R^{2B} are hydrogen, and R⁴ is C₃-C₈ cycloalkyl or phenyl; each of which is optionally substituted by 1, 2, or 3 substituents independently selected from the group consisting of C₁-C₈ alkyl, halogen, C₁-C₆ haloalkyl, and C₃-C₆ cycloalkyl.

Accordingly, in certain embodiments, R^{1A} and R^{1B} are methyl. In certain embodiments, R^{1A} and R^{1B} are C₁-C₃ alkyl.

In certain embodiments, R^{2A} and R^{2B} are hydrogen.

In certain embodiments, Y is a bond. In certain embodiments, Y is C₁-C₆ alkylene.

In certain embodiments, R⁴ is C₃-C₈ cycloalkyl optionally substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, and —(C₂-C₄ alkynyl)-C₁-C₆ alkoxy. In certain embodiments, R⁴ is C₃-C₈ cycloalkyl optionally substituted by 1, 2, or 3 substituents independently selected from the group consisting of C₁-C₆ alkyl, C₁-C₆ haloalkyl, C₃-C₆ cycloalkyl, C₁-C₆ alkoxy, and C₂-C₄ alkynyl. In certain embodiments, R⁴ is C₄-C₆ cycloalkyl substituted by 1, 2, or 3 substituents independently selected from the group consisting of C₁-C₆ alkyl, C₁-C₆ haloalkyl, C₃-C₆ cycloalkyl, and C₁-C₆ alkoxy. In certain embodiments, R⁴ is C₄-C₆ cycloalkyl optionally substituted by 1 or 2 substituents independently selected from the group consisting of C₁-C₆ alkyl, C₁-C₆ haloalkyl, C₃-C₆ cycloalkyl, and C₁-C₆ alkoxy.

In certain embodiments, R⁴ is a partially unsaturated 9-10 membered bicyclic carbocyclyl optionally substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, and —(C₂-C₄ alkynyl)-C₁-C₆ alkoxy. In certain embodiments, R⁴ is a partially unsaturated 9-10 membered bicyclic carbocyclyl optionally substituted by C₁-C₆ alkyl.

In certain embodiments, R⁴ is phenyl substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, and —(C₂-C₄ alkynyl)-C₁-C₆ alkoxy. In certain embodiments, R⁴ is phenyl substituted by 1, 2, or 3 substituents independently selected from the group consisting of C₁-C₆ alkyl, halogen, C₁-C₆ haloalkyl, C₃-C₆ cycloalkyl, C₁-C₆ alkoxy, and C₂-C₄ alkynyl. In certain embodiments, R⁴ is phenyl substituted by 1, 2, or 3 substituents independently selected from the group consisting of C₁-C₆ alkyl, halogen, C₁-C₆ haloalkyl, C₃-C₆ cycloalkyl, C₂-C₄ alkynyl, and —(C₂-C₄ alkynyl)-C₁-C₆ alkoxy. In certain embodiments, R⁴ is C₃-C₈ cycloalkyl or phenyl; each of which is optionally substituted by 1, 2, or 3 substituents independently selected from the group consisting of C₁-C₈ alkyl, halogen, C₁-C₆ haloalkyl, and C₃-C₆ cycloalkyl.

The description above describes multiple embodiments relating to compounds of Formula II-A. The patent application specifically contemplates all combinations of the embodiments.

In certain other embodiments, the compound is one of the compounds listed in Table 3 below or a pharmaceutically acceptable salt thereof. In certain other embodiments, the compound is one of the compounds listed in Table 4 or a pharmaceutically acceptable salt thereof. In certain other embodiments, the compound is a compound in any one of Tables 1, 2, 3, or 4 herein, or a pharmaceutically acceptable salt thereof. In certain other embodiments, the compound is one of the compounds in the Examples or a pharmaceutically acceptable salt thereof. In certain other embodiments, the compound is one of the compounds listed in Table 5 or a pharmaceutically acceptable salt thereof.

TABLE 3

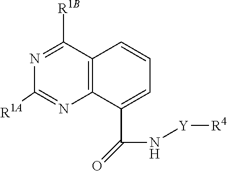

| Compound | | | | |
|---|---|---|---|---|
| No. | R^{1A} | R^{1B} | Y | R⁴ |
| III-1 | —CH₃ | —OCH₃ | a bond | 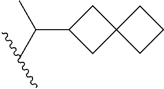 [SMILES: *C(C)C1CC2(CCC2)C1] |
| III-2 | —OCH₃ | —CH₃ | a bond | 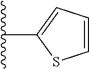 [SMILES: *c1cccs1] |
| III-3 | —CH₃ | —N(H)CH₃ | a bond | 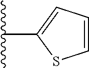 [SMILES: *c1cccs1] |
| III-4 | —N(H)CH₃ | —CH₃ | a bond | 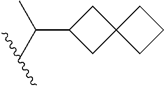 [SMILES: *C(C)C1CC2(CCC2)C1] |
| III-5 | —CH₃ | —CH₃ | a bond | 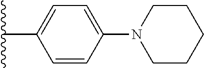 [SMILES: *c1ccc(N2CCCCC2)cc1] |
| III-6 | —CH₃ | —CH₃ | a bond | 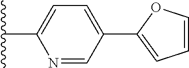 [SMILES: *c1ccc(-c2ccco2)cn1] |
| III-7 | —CH₃ | —CH₃ | a bond | 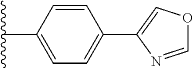 [SMILES: *c1ccc(-c2cocn2)cc1] |
| III-8 | —CH₃ | —CH₃ | a bond | 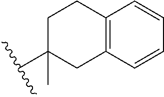 [SMILES: *C1(C)CCc2ccccc2C1] |
| III-9 | —N(H)CH₃ | —CH₃ | a bond | 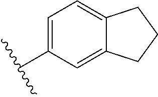 [SMILES: *c1ccc2c(c1)CCC2] |
| III-10 | —CH₃ | —CH₃ | a bond | 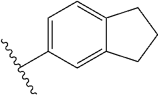 [SMILES: *c1ccc2c(c1)CCC2] |
| III-11 | —OCH₃ | —CH₃ | a bond | 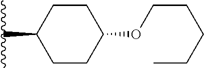 [SMILES: *[C@H]1CC[C@H](OCCCCC)CC1] |
| III-12 | —N(H)CH₃ | —CH₃ | a bond | 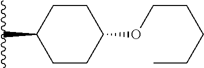 [SMILES: *[C@H]1CC[C@H](OCCCCC)CC1] |
| III-13 | —CH₃ | —CH₃ | a bond | 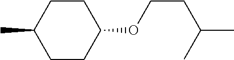 |
| III-14 | —CH₃ | —CH₃ | a bond | 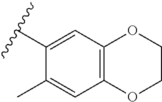 [SMILES: *c1cc2c(cc1C)OCCO2] |
| III-15 | —CH₃ | —CH₃ | C₁-C₄ alkylene | 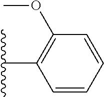 [SMILES: *c1ccccc1OC] |
| III-16 | —CH₃ | —CH₃ | C₁-C₄ alkylene | 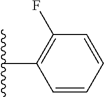 [SMILES: *c1ccccc1F] |
| III-17 | —CH₃ | —CH₃ | C₁-C₄ alkylene | 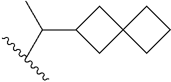 [SMILES: *C(C)C1CC2(CCC2)C1] |
| III-18 | —CH₃ | —CH₃ | C₁-C₄ alkylene | 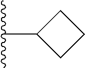 [SMILES: *C1CCC1] |
| III-19 | —CH₃ | —CH₃ | C₁-C₄ alkylene | 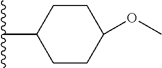 [SMILES: *C1CCC(OC)CC1] |

Methods for preparing compounds described herein are illustrated in the following synthetic scheme. These scheme is provided for the purpose of illustrating the invention, and should not be regarded in any manner as limiting the scope or the spirit of the invention. Starting materials shown in the scheme can be obtained from commercial sources or can be prepared based on procedures described in the literature.

The synthetic route illustrated in Scheme 1 depicts an exemplary procedure for preparing substituted pyrrolo[1,2-a][1,3,5]triazine-8-carboxamides. In the first step condensation of (E)-N-(1-(dimethylamino)ethylidene)acetamide (Rⁱⁱ=Rⁱⁱⁱ=Me) and ethyl 2-amino-1H-pyrrole-3-carboxylate (Rⁱ=H) A in acetic acid at high temperature afford the corresponding triazine, 2,4-dimethylpyrrolo[1,2-a][1,3,5]triazine-8-carboxylic acid B (Rⁱ=H, Rⁱⁱ=Rⁱⁱⁱ=Me). Hydrolysis of carboxylic ester B under mild, neutral conditions provides carboxylic acid C. In the final step, coupling of carboxylic acid C with a variety of substituted aromatic or aliphatic amines may be accomplished using standard peptide coupling procedures (such as HATU and/or HOBT in DMF in the presence of DIPEA) to afford carboxamide D.

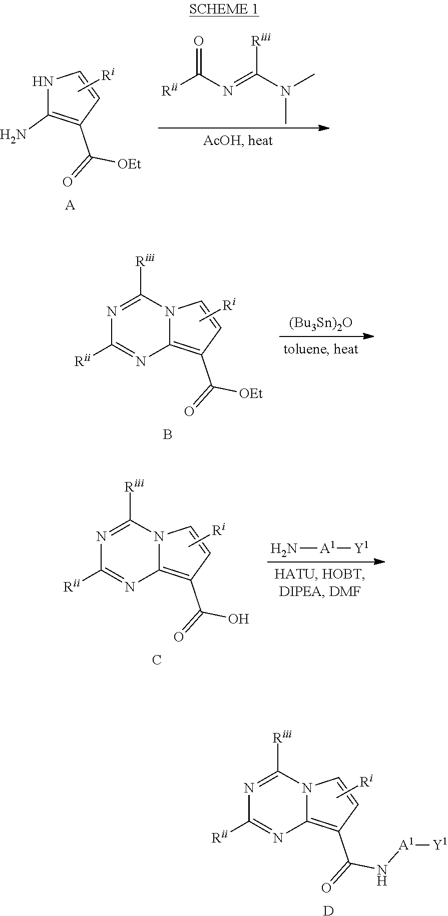

The synthetic route illustrated in Scheme 2 depicts an exemplary procedure for preparing substituted quinazolinyl-8-carboxamides. In the first step condensation of acetic anhydride (Rⁱ=Me) and 2-amino-3-bromobenzoic acid (Rⁿ=H) E at high temperature followed by reaction with NH₄OAc affords 8-bromo-2-methylquinazolin-4(3H)-one (Rⁱ=Me, Rⁿ=H) F. Conversion of bromide F to the corresponding carboxylate mat be accomplished using Pd(0) catalyzed carbonylation, followed by reaction with POCl₃ to afford methyl 4-chloro-2-methylquinazoline-8-carboxylate (Rⁱ=Me, Rⁿ=H), which is subjected to Pd(0)-catalyzed coupling with Rⁱⁱ—X to affored methyl ester G. Base hydrolysis of ester G provides methyl 2,4-dimethylquinazoline-8-carboxylate (Rⁱ=Rⁱⁱ=Me, Rⁿ=H) H. In the final step, coupling of carboxylic acid H with a variety of substituted aromatic or aliphatic amines may be accomplished using standard peptide coupling procedures (such as HATU and/or HOBT in DMF in the presence of DIPEA) to afford carboxamide I.

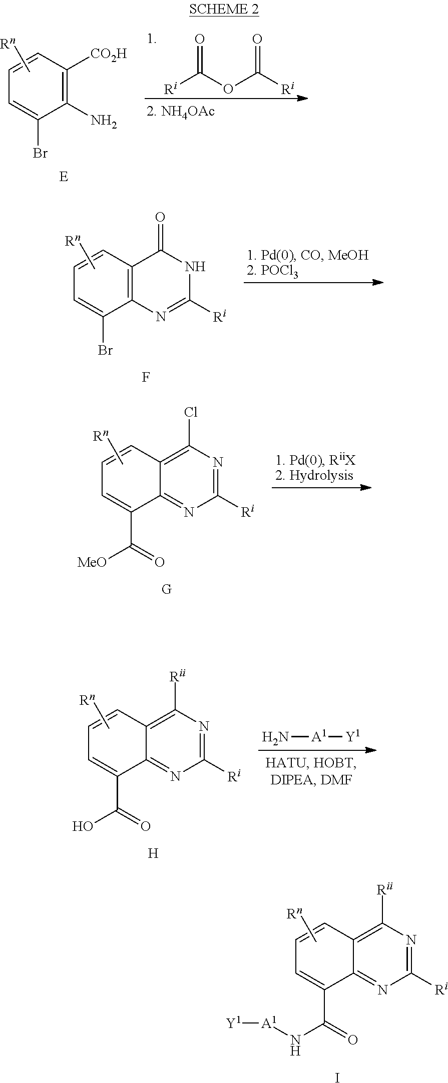

The reaction procedures in Schemes 1 and 2 are contemplated to be amenable to preparing a wide variety of carboxamide compounds having different substituents at variable R. Furthermore, if a functional group that is part of variable R would not be amenable to a reaction condition described in Schemes 1 and 2, it is contemplated that the functional group can first be protected using standard protecting group chemistry and strategies, and then the protecting group is removed after completing the desired synthetic transformation. See, for example, Greene, T. W.; Wuts, P. G. M. Protective Groups in Organic Synthesis, 2^{nd} ed.; Wiley: New York, 1991, for further description of protecting chemistry and strategies. In certain other embodiments, a functional group in substituent R can converted to another functional group using standard functional group manipulation procedures known in the art. See, for example, “Comprehensive Organic Synthesis” (B. M. Trost & I. Fleming, eds., 1991-1992).

### III. Therapeutic Applications

The invention provides methods of treating medical disorders, such as Gaucher disease, Parkinson's disease, Lewy body disease, dementia, multiple system atrophy, epilepsy, bipolar disorder, schizophrenia, an anxiety disorder, major depression, polycystic kidney disease, type 2 diabetes, open angle glaucoma, multiple sclerosis, endometriosis, and multiple myeloma, using the substituted pyrrolo[1,2-a]triazine compounds, related compounds, and pharmaceutical compositions described herein. Treatment methods include the use of substituted pyrrolo[1,2-a]triazine compounds and related compounds described herein as stand-alone therapeutic agents and/or as part of a combination therapy with another therapeutic agent. Although not wishing to be bound by a particular theory, it is understood that substituted pyrrolo[1,2-a]triazine compounds and related compounds described herein may activate glucocerebrosidase (Gcase).

### Methods of Treating Medical Disorders

One aspect of the invention provides a method of treating a disorder selected from the group consisting of Gaucher disease, Parkinson's disease, Lewy body disease, dementia, multiple system atrophy, epilepsy, bipolar disorder, schizophrenia, an anxiety disorder, major depression, polycystic kidney disease, type 2 diabetes, open angle glaucoma, multiple sclerosis, endometriosis, and multiple myeloma. The method comprises administering to a patient in need thereof a therapeutically effective amount of a substituted pyrrolo[1,2-a]triazine compound or related compound described herein to treat the disorder. The compound may be a compound of Formula I, which, as described above in Section II, is represented by:

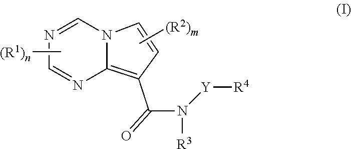

or a pharmaceutically acceptable salt thereof, wherein:

R¹ and R² each represent independently for each occurrence hydrogen, C₁-C₆ alkyl, halogen, C₁-C₆ haloalkyl, C₃-C₆ cycloalkyl, C₁-C₆ alkoxy, —(C₁-C₄ alkylene)-O—(C₁-C₆ alkyl), —(C₁-C₄ alkylene)-O—(C₃-C₆ cycloalkyl), 3-6 membered heterocyclyl, 6-membered aryl, cyano, or —N(R⁵)₂;

R³ is hydrogen, C₁-C₆ alkyl, or C₃-C₆ cycloalkyl;

R⁴ is one of the following:
C₃-C₈ cycloalkyl, 3-8 membered heterocycloalkyl, 9-13 membered spiroheterocycloalkyl, —(C₂-C₆ alkylene)-O-phenyl, phenyl, heteroaryl, a partially unsaturated 9-10 membered bicyclic carbocyclyl, or a partially unsaturated 8-10 membered bicyclic heterocyclyl; each of which is optionally substituted by 1, 2, or 3 substituents independently selected from the group consisting of C₁-C₈ alkyl, halogen, C₁-C₆ haloalkyl, C₃-C₆ cycloalkyl, —(C₁-C₆ alkylene,)—(C₃-C₆ cycloalkyl), hydroxyl, C₁-C₆ alkoxy, —O—(C₃-C₆ cycloalkyl), C₂-C₄ alkynyl, —(C₂-C₄ alkynyl)-C₁-C₆ alkoxy, aryl, —O-aryl, heteroaryl, saturated 3-8 membered heterocyclyl, amino, and —CO₂R⁵; or
C₁-C₆ alkyl;

R⁵ represents independently for each occurrence hydrogen, C₁-C₆ alkyl, or C₃-C₆ cycloalkyl;

Y is a bond, —C(O)—, C₁-C₆ alkylene, C₁-C₆ haloalkylene, or C₃-C₆ cycloalkylene; and

n and m are independently 1 or 2.

In certain embodiments, the disorder is Gaucher disease, Parkinson's disease, Lewy body disease, dementia, or multiple system atrophy. In certain other embodiments, the disorder is Gaucher disease. In certain embodiments, the disorder is Parkinson's disease. In certain embodiments, the disorder is Lewy body disease. In certain embodiments, the disorder is dementia. In certain embodiments, the disorder is a dementia selected from the group consisting of Alzheimer's disease, frontotemporal dementia, and a Lewy body variant of Alzheimer's disease. In certain embodiments, the disorder is multiple system atrophy.

In certain embodiments, the disorder is an anxiety disorder, such as panic disorder, social anxiety disorder, or generalized anxiety disorder.

Efficacy of the compounds in treating Gaucher disease, Parkinson's disease, Lewy body disease, dementia, multiple system atrophy, epilepsy, bipolar disorder, schizophrenia, an anxiety disorder, major depression, polycystic kidney disease, type 2 diabetes, open angle glaucoma, multiple sclerosis, endometriosis, and multiple myeloma may be evaluated by testing the compounds in assays known in the art for evaluating efficacy against these diseases and/or, e.g., for activation of glucocerebrosidase (Gcase), as discussed in the Examples below.

In certain embodiments, the patient is a human.

In certain embodiments, the compound is one of the generic or specific compounds described in Section II, such as a compound of Formula I, a compound embraced by one of the further embodiments describing definitions for certain variables of Formula I, a compound of Formula I-A, or a compound embraced by one of the further embodiments describing definitions for certain variables of Formula I-A.

The description above describes multiple embodiments relating to methods of treating various disorders using certain substituted pyrrolo[1,2-a]triazine compounds and related compounds. The patent application specifically contemplates all combinations of the embodiments. For example, the invention contemplates methods for treating Gaucher disease, Parkinson's disease, Lewy body disease, dementia, or multiple system atrophy by administering a therapeutically effective amount of a compound of Formula I-A wherein R^{1A} and R^{1B} are C₁-C₃ alkyl, R^{2A} and R^{2B} are hydrogen, and R⁴ is C₃-C₈ cycloalkyl or phenyl; each of which is optionally substituted by 1, 2, or 3 substituents independently selected from the group consisting of C₁-C₈ alkyl, halogen, C₁-C₆ haloalkyl, and C₃-C₆ cycloalkyl.

### Medical Use and Preparation of Medicament

Another aspect of the invention relates to compounds and compositions described herein for use in treating a disorder described herein. Another aspect of the invention pertains to use of a compound or composition described herein in the preparation of a medicament for treating a disorder described herein.

### Combination Therapy

The invention embraces combination therapy, which includes the administration of a substituted pyrrolo[1,2-a]triazine compound or related compound described herein (such as compound of Formula I or I-A) and a second agent as part of a specific treatment regimen intended to provide the beneficial effect from the co-action of these therapeutic agents. The beneficial effect of the combination may include pharmacokinetic or pharmacodynamic co-action resulting from the combination of therapeutic agents.

Exemplary second agents for use in treating Gaucher disease include, for example, taliglucerase alfa, velaglucerase alfa, eliglustat, and miglustat. Exemplary second agents for use in treating Parkinson's disease include, for example, a glucosylceramide synthase inhibitor (e.g., ibiglustat), an acid ceramidase inhibitor (e.g., carmofur), an acid shingomyelinase activator, or salt thereof. Additional glucosylceramide synthase inhibitors for use in combination therapies include, for example, those described in International Patent Application Publications WO 2015/089067, WO 2014/151291, WO 2014/043068, WO 2008/150486, WO 2010/014554, WO 2012/129084, WO 2011/133915, and WO 2010/091164; U.S. Pat. Nos. 9,126,993, 8,961,959, 8,940,776, 8,729,075, and 8,309,593; and U.S. Patent Application Publications US 2014/0255381 and US 2014/0336174; each of which are hereby incorporated by reference. Additional acid ceramidase inhibitors for use in combination therapies include, for example, those described in International Patent Application Publications WO 2015/173168 and WO 2015/173169, each of which are hereby incorporated by reference.

### IV. Pharmaceutical Compositions

The invention provides pharmaceutical compositions comprising a substituted pyrrolo[1,2-a]triazine compound or related compound described herein, such as a compound of Formula I or I-A. In certain embodiments, the pharmaceutical compositions preferably comprise a therapeutically-effective amount of one or more of the substituted pyrrolo[1,2-a]triazine compounds or related compounds described above, formulated together with one or more pharmaceutically acceptable carriers. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration.

The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and

a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

### V. Kits for Use in Medical Applications

Another aspect of the invention provides a kit for treating a disorder. The kit comprises: i) instructions for treating a medical disorder, such as Gaucher disease, Parkinson's disease, Lewy body disease, dementia, or multiple system atrophy; and ii) a substituted pyrrolo[1,2-a]triazine compound or related compound described herein, such as a compound of Formula I or I-A. The kit may comprise one or more unit dosage forms containing an amount of a substituted pyrrolo[1,2-a]triazine compound or related compound described herein, such as a compound of Formula I, that is effective for treating said medical disorder, e.g., Gaucher disease, Parkinson's disease, Lewy body disease, dementia, or multiple system atrophy.

The description above describes multiple aspects and embodiments of the invention, including substituted pyrrolo[1,2-a]triazine compounds, related compounds, compositions comprising a substituted pyrrolo[1,2-a]triazine compound or related compound, methods of using the substituted pyrrolo[1,2-a]triazine compounds or related compounds, and kits. The patent application specifically contemplates all combinations and permutations of the aspects and embodiments. For example, the invention contemplates treating Gaucher disease, Parkinson's disease, Lewy body disease, dementia, or multiple system atrophy in a human patient by administering a therapeutically effective amount of a compound of Formula I-A. Further, for example, the invention contemplates a kit for treating Gaucher disease, Parkinson's disease, Lewy body disease, dementia, or multiple system atrophy, the kit comprising (i) instructions for treating Gaucher disease, Parkinson's disease, Lewy body disease, dementia, or multiple system atrophy and (ii) a substituted pyrrolo[1,2-a]triazine compound described herein, such as a compound of Formula I-A.

### EXAMPLES

The invention now being generally described, will be more readily understood by reference to the following examples, which are included merely for purposes of illustration of certain aspects and embodiments of the present invention, and are not intended to limit the invention.

### Example 1—Preparation of Pyrrolo[1,2-a]triazine & Related Compounds

Pyrrolo[1,2-a]triazine compounds and related compounds were prepared based on general procedures described in Part I below. Exemplary procedures for preparing specific carboxylic acid compounds useful as synthetic intermediates in the preparation of certain substituted pyrrolo[1,2-a]triazine compounds and related compounds are provided in Part II below. Specific pyrrolo[1,2-a]triazine compounds and related compounds prepared according to the general procedures are provided in Part III below.

### Part I—General Procedures

General Procedure A: Preparation of Amide by Coupling of a Carboxylic Acid Compound with an Amine Compound

To a stirred solution of carboxylic acid compound (1.0 equivalent), HATU (1.5 equivalents), and DIPEA (3.75 equivalents) in DCM or DMF (˜4 mL/0.2 mmol) is added amine compound (1.25-2.0 equivalents). The reaction mixture is stirred at room temperature for 4-16 hours, and then washed with saturated aqueous NaHCO₃ solution (5 mL/0.2 mmol), aqueous citric acid solution (5 mL/0.2 mmol) and brine (5 mL/0.2 mmol). The combined extracts are dried over anhydrous Na₂SO₄, filtered and concentrated in vacuo. The resulting crude material is purified by silica gel column chromatography or preparatory HPLC to give the amide compound.

### General Procedure B: Conversion of Carboxylic Ester Compound to Carboxylic Acid Compound

To a solution of carboxylic ester (1.0 equivalent) in EtOH (5.0 mL/1.0 mmol) and water (0-3.0 mL/1.0 mmol) is added NaOH (2.0-5.0 equivalents) and the mixture is heated at 80° C. for 2 hours and then concentrated. To the concentrate, 6N HCl solution is added to adjust the pH to 5-6 and then the mixture is stirred for 10 minutes and subsequently filtered. The resulting solid is collected and dried to give the carboxylic acid compound.

### General Procedure C: Preparation of Coupled Aryl and Heteroaryl Groups Using Suzuki Catalyzed Coupling Conditions Between an Organoboronic Acid or Ester and an Aryl Halide or Heteroaryl Halide

A suspension of heteroaryl chloride (1 equivalent), organoboronic acid or organoboronic ester (1.2 equivalents), K₃PO₄ (3.0 equivalents) and Pd(dppf)Cl₂.DCM (5 mol %) or Pd₂(dba)₃ (10 mol %) in DME or 1,4-dioxane (40 mL/mmol) is stirred at 70-100° C. for 2-6 hours under N₂. Then, the reaction mixture is quenched with water (30 mL/mmol) and resulting mixture extracted with EtOAc (30 mL/mmol x 3). The organic phases are washed with water (30 mL/mmol) and brine (30 mL/mmol), dried over anhydrous Na₂SO₄ and filtered. The filtrate is concentrated in vacuo, and the resulting residue is purified by silica gel column chromatography to afford the coupled ring system.

### Part II—Preparation of Specific Carboxylic Acid Compounds

Exemplary procedures for preparing specific carboxylic acid compounds useful in the preparation of certain substituted carboxamide compounds are provided below.

### 2,4-Dimethylpyrrolo[1,2-a][1,3,5]triazine-8-carboxylic acid

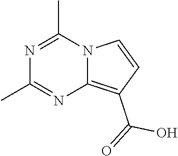

A suspension of acetamide (590 mg, 10 mmol) and 1,1-dimethoxy-N,N-dimethylethan-1-amine (1.33 g, 10 mmol) in toluene (20 mL) was heated to reflux for 2 h followed by the addition of a solution of ethyl 2-amino-1H-pyrrole-3-carboxylate (1.54 g, 10 mmol) in acetic acid (2 mL). The reaction mixture was stirred at reflux for 4 h, then cooled to RT and concentrated in vacuo. The resulting residue was purified by silica gel chromatography (PE/EA; 1/2) to give ethyl 2,4-dimethylpyrrolo[1,2-a][1,3,5]triazine-8-carboxylate (780 mg, 36%) as a yellow solid. ¹H NMR (500 MHz, DMSO-d₆) δ 7.54 (d, J=3.5 Hz, 1H), 7.26 (d, J=3.5 Hz, 1H), 4.26 (d, J=7.0 Hz, 2H), 2.76 (s, 3H), 2.55 (s, 3H), 1.30 (t, J=7.0 Hz, 3H). LC-MS m/z: 220.0 [M+H]⁺. LC-MS Purity (214 nm): 96%; t_{R}=1.43 min.

To a stirred solution of bis(tributyl)tin oxide (1.60 mL, 3.1 mmol) in toluene (10 mL) was added ethyl 2,4-dimethylpyrrolo[1,2-a][1,3,5]triazine-8-carboxylate (340 mg, 1.55 mmol). The mixture was refluxed for 2 days, and then concentrated in vacuo. The resulting oil was dissolved in EtOAc (10 mL) and extracted with 5% aqueous NaHCO₃ (3×5 mL). The aqueous phase was acidified to pH 4-5 with dilute HCl and concentrated in vacuo. The resulting residue was dissolved with DCM/MeOH (10/1 50 mL) and filtered. The filtrate was concentrated to afford 2,4-dimethylpyrrolo[1,2-a][1,3,5]triazine-8-carboxylic acid (200 mg, 67%) as a white solid. LC-MS m/z: 192.1 [M+H]⁺; t_{R}=0.4 min.

### 2,4-Dimethylquinazoline-8-carboxylic acid

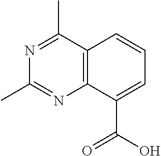

A mixture of 2-amino-3-bromobenzoic acid (5 g, 23.3 mol) and acetic anhydride (20 mL) was heated at 140° C. under N₂ for 3 h, cooled and concentrated in vacuo. The resulting residue was dissolved in toluene and concentrated in vacuo. This procedure was repeated twice to afford 8-bromo-2-methyl-4H-benzo[d][1,3]oxazin-4-one (5.58 g, 100%) as a yellow solid. LC-MS: m/z 240.2 [M+H]⁺. Purity (254 nm): 91.6%; t_{R}=1.91 min.

A mixture of 8-bromo-2-methyl-4H-benzo[d][1,3]oxazin-4-one (5.56 g, 23.3 mol) and ammonium acetate (18 g, 233 mol) was heated at 140° C. under N₂ for 4 h, and cooled to RT, followed by the addition of 150 mL of water under intensive stirring. The resulting precipitate was collected by filtration and washed three times with 50 mL of water. The product was dried in vacuo to afford 8-bromo-2-methylquinazolin-4(3H)-one (4.5 g, 81%) as a yellow solid. LC-MS: m/z 239.1 [M+H]⁺. Purity (214 nm): >99%; t_{R}=1.63 min.

To a solution of 8-bromo-2-methylquinazolin-4(3H)-one (4.5 g, 18.9 mmol) in MeOH (150 mL) and MeCN (75 mL) was added Et₃N (9.54 g, 94.5 mmol) and PdCl₂(dppf).DCM (771 mg, 0.94 mmol). The mixture was placed under 20 bar of CO, heated to 100° C., and stirred vigorously for 18 h. The reaction mixture was cooled to ambient temperature and filtered over celite and the filtrate was concentrated in vacuo. This residue was purified by flash column chromatography (5/1, EA/PE) to afford methyl 2-methyl-4-oxo-3,4-dihydroquinazoline-8-carboxylate (2.9 g, 70%) as a pale yellow solid. ¹H NMR (500 MHz, DMSO-d₆) δ 12.37 (s, 1H), 8.21 (dd, J=8.0 Hz, 1.5 Hz, 1H), 7.92 (dd, J=8.0 Hz, 1.5 Hz, 1H), 7.50 (t, J=8.0 Hz, 1H), 3.86 (s, 3H), 2.35 (s, 3H). LC-MS: m/z 219.2 [M+H]⁺. Purity (214 nm): >99%; t_{R}=1.46 min.

To a solution of methyl 2-methyl-4-oxo-3,4-dihydroquinazoline-8-carboxylate (1.7 g, 7.80 mmol) in POCl₃ (20 mL) was added DIEA (2 mL). The reaction mixture was refluxed for 4 h, cooled to RT, and added slowly to a saturated solution of NaHCO₃ in an ice bath. The mixture was extracted with EA (150 mL×2). The combined extracts were washed with water (50 mL×2) and brine (50 mL×2), dried over anhydrous Na₂SO₄, and filtered. The filtrate was concentrated in vacuo, and the resulting residue was purified by column chromatography (EA/PE=1/1) to afford methyl 4-chloro-2-methylquinazoline-8-carboxylate (1.0 g, 54%) as a yellow solid. ¹H NMR (500 MHz, CDCl₃) δ 8.39 (dd, J=8.0 Hz, 1.5 Hz, 1H), 8.24 (dd, J=8.0 Hz, 1.5 Hz, 1H), 7.68 (t, J=8.0 Hz, 1H), 4.05 (s, 3H), 2.89 (s, 3H). LC-MS: m/z 237.1 [M+H]⁺. Purity (214 nm): >99%; t_{R}=1.89 min.

To a solution of methyl 4-chloro-2-methylquinazoline-8-carboxylate (1 g, 4.24 mmol) in DMF (40 mL) was added 2,4,6-trimethyl-1,3,5,2,4,6-trioxatriborinane (534 mg, 4.24 mmol), Na₂CO₃ (1123 mg, 10.6 mmol), and Pd(PPh₃)₄(485 mg, 0.42 mmol). The mixture was purged three times with N₂, stirred at 80° C. for 6 hours, cooled to RT, and diluted with water (100 mL). The mixture was extracted with EA (50 mL×3), washed with brine (50 mL×2), dried over anhydrous Na₂SO₄, and filtered. The filtrate was concentrated in vacuo, and the resulting residue was purified by flash column chromatography (20% to 50% EA/PE) to afford methyl 2,4-dimethylquinazoline-8-carboxylate (680 mg, 74%) as a yellow solid. LC-MS: m/z 217.2 [M+H]⁺. Purity (254 nm): >99%; t_{R}=1.63 min.

Following general procedure B, methyl 2,4-dimethylquinazoline-8-carboxylate (680 mg, 3.15 mmol) afforded 2,4-dimethylquinazoline-8-carboxylic acid (520 mg, 82%) as a yellow solid. LC-MS: m/z 203.2 [M+H]⁺. Purity (214 nm): 94%; t_{R}=0.31 min.

### Part III—Preparation of Substituted pyrrolo[1,2-a]triazine Compounds 2,4-Dimethyl-N-(1-methyl-1,2,3,4-tetrahydronaphthalen-1-yl)pyrrolo[1,2-a][1,3,5]triazine-8-carboxamide

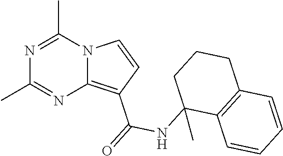 [SMILES: Cc1nc(C)n2ccc(C(=O)NC3(C)CCCc4ccccc43)c2n1]

Following general procedure A, 2,4-dimethylpyrrolo[1,2-a][1,3,5]triazine-8-carboxylic acid (50 mg, 0.28 mmol) and 1-methyl-1,2,3,4-tetrahydronaphthalen-1-amine afforded the title compound (9.6 mg, 11%) as a yellow solid. ¹H NMR (400 MHz, MeOD) δ 8.94 (s, 1H), 7.50 (d, J=7.2 Hz, 1H), 7.42 (d, J=3.6 Hz, 1H), 7.29 (d, J=3.6 Hz, 1H), 7.19-7.10 (m, 3H), 2.98-2.86 (m, 1H), 2.87-2.71 (m, 5H), 2.53 (s, 3H), 2.08-2.01 (m, 1H), 1.97-1.87 (m, 2H), 1.79 (s, 3H). LC-MS m/z: 335.7 [M+H]⁺. HPLC Purity (214 nm): >99%; t_{R}=9.26 min.

### N-(2,3-Dihydro-1H-inden-5-yl)-2,4-dimethylpyrrolo[1,2-a][1,3,5]triazine-8-carboxamide

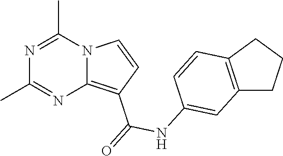 [SMILES: Cc1nc(C)n2ccc(C(=O)Nc3ccc4c(c3)CCC4)c2n1]

Following general procedure A, 2,4-dimethylpyrrolo[1,2-a][1,3,5]triazine-8-carboxylic acid (50 mg, 0.28 mmol) and 2,3-dihydro-1H-inden-5-amine afforded the title compound (7.2 mg, 9%) as a white solid. ¹H NMR (400 MHz, MeOD-d₄) δ 7.59 (s, 1H), 7.49 (d, J=4.0 Hz, 1H), 7.44 (d, J=4.0 Hz, 1H), 7.38 (d, J=8.0 Hz, 1H), 7.20 (d, J=8.0 Hz, 1H), 2.96-2.81 (m, 7H), 2.72 (s, 3H), 2.16-2.07 (m, 2H). LC-MS m/z: 307.7 [M+H]⁺. HPLC Purity (214 nm): >99%; t_{R}=9.34 min.

### 2,4-Dimethyl-N-(1-phenylpropyl)pyrrolo[1,2-a][1,3,5]triazine-8-carboxamide

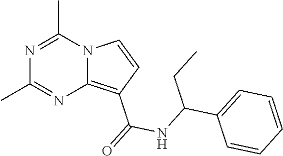 [SMILES: CCC(NC(=O)c1ccn2c(C)nc(C)nc12)c1ccccc1]

Following general procedure A, 2,4-dimethylpyrrolo[1,2-a][1,3,5]triazine-8-carboxylic acid (50 mg, 0.28 mmol) and 1-phenylpropan-1-amine afforded the title compound (10.3 mg, 13%) as a yellow solid. ¹H NMR (400 MHz, MeOD-d₄) δ 7.48-7.29 (m, 6H), 7.31-7.10 (m, 1H), 5.06 (t, J=6.4 Hz, 1H), 3.32 (dt, J=3.2, 1.6 Hz, 1H), 2.85-2.70 (m, 2H), 2.66 (s, 3H), 2.10-1.86 (m, 2H), 0.98 (t, J=7.2 Hz, 3H). LC-MS m/z: 309.7 [M+H]⁺. HPLC Purity (214 nm): 98.2%; t_{R}=8.61 min.

### 2,4-Dimethyl-N-((1S,4S)-4-(pentyloxy)cyclohexyl) pyrrolo[1,2-a][1,3,5]triazine-8-carboxamide

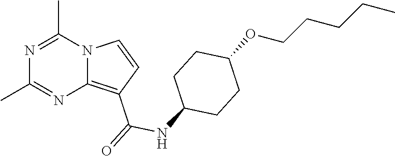 [SMILES: CCCCCO[C@H]1CC[C@H](NC(=O)c2ccn3c(C)nc(C)nc23)CC1]

Following general procedure A, 2,4-dimethylpyrrolo[1,2-a][1,3,5]triazine-8-carboxylic acid (25 mg, 0.28 mmol) and (1S,4S)-4-(pentyloxy)cyclohexan-1-amine afforded the title compound (7.5 mg, 16%) as a yellow solid. ¹H NMR (500 MHz, DMSO-d₆) δ 8.04 (d, J=7.5 Hz, 1H), 7.54 (d, J=3.5 Hz, 1H), 7.25 (d, J=3.5 Hz, 1H), 3.80-3.79 (m, 1H), 3.40-3.36 (m, 2H), 3.29-3.28 (m, 1H), 2.77 (s, 3H), 2.57 (s, 3H), 1.97-1.95 (m, 4H), 1.49-1.45 (m, 2H), 1.38-1.23 (m, 8H), 0.86 (t, J=8.0 Hz, 3H). LC-MS m/z: 359.7 [M+H]⁺. HPLC Purity (214 nm): >99%; t_{R}=9.65 min.

### N-(4-Ethynylphenyl)-2,4-dimethylpyrrolo[1,2-a][1,3,5]triazine-8-carboxamide

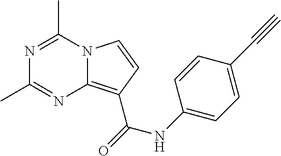 [SMILES: C#Cc1ccc(NC(=O)c2ccn3c(C)nc(C)nc23)cc1]

Following general procedure A, 2,4-dimethylpyrrolo[1,2-a][1,3,5]triazine-8-carboxylic acid (50 mg, 0.26 mmol) and 4-ethynylaniline afforded the title compound (7.7 mg, 10%) as a brown solid. ¹H NMR (500 MHz, CDCl₃) δ 10.33 (s, 1H), 7.73 (d, J=9.0 Hz, 2H), 7.57 (d, J=3.0 Hz, 1H), 7.50 (d, J=8.0 Hz, 2H), 7.15 (d, J=3.5 Hz, 1H), 3.05 (s, 1H), 2.81 (s, 3H), 2.74 (s, 3H). LC-MS m/z: 291.2 [M+H]⁺. HPLC: Purity (214 nm): 92%; t_{R}=9.93 min.

### N-(2-Cyclopropylpropan-2-yl)-2,4-dimethylpyrrolo[2-a][1,3,5]triazine-8-carboxamide

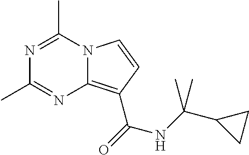 [SMILES: Cc1nc(C)n2ccc(C(=O)NC(C)(C)C3CC3)c2n1]

Following general procedure A, 2,4-dimethylpyrrolo[1,2-a][1,3,5]triazine-8-carboxylic acid (50 mg, 0.26 mmol) and 2-cyclopropylpropan-2-amine afforded the title compound (9 mg, 13%) as a gray solid. ¹H NMR (500 MHz, DMSO-d₆) δ 8.10 (s, 1H), 7.53 (d, J=3.5 Hz, 1H), 7.21 (d, J=3.5 Hz, 1H), 2.77 (s, 3H), 2.56 (s, 3H), 1.34 (s, 6H), 1.34-1.30 (m, 1H), 0.44 (d, J=6.5 Hz, 4H). LC-MS m/z: 273.3 [M+H]⁺. HPLC: Purity (214 nm): 97%; t_{R}=9.66 min.

### N-(1-Cyclopropyl-2,2,2-trifluoroethyl)-2,4-dimethylpyrrolo[1,2-a][1,3,5]triazine-8-carboxamide

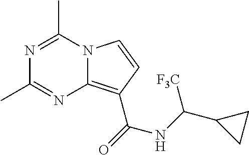 [SMILES: Cc1nc(C)n2ccc(C(=O)NC(C3CC3)C(F)(F)F)c2n1]

Following general procedure A, 2,4-dimethylpyrrolo[1,2-a][1,3,5]triazine-8-carboxylic acid (100 mg, 0.52 mmol) and 1-cyclopropyl-2,2,2-trifluoroethanamine hydrochloride afforded the title compound (61 mg, 38%) as a gray solid. ¹H NMR (500 MHz, DMSO-d₆) δ 8.46 (d, J=9.5 Hz, 1H), 8.60 (d, J=3.5 Hz, 1H), 8.30 (d, J=3.5 Hz, 1H), 4.48-4.42 (m, 1H), 2.79 (s, 3H), 2.59 (s, 3H), 1.26-1.22 (m, 1H), 0.68-0.61 (m, 1H), 0.60-0.55 (m, 1H), 0.52-0.48 (m, 1H), 0.40-0.36 (m, 1H). LC-MS m/z: 313.2 [M+H]⁺. HPLC: Purity (214 nm): 99%; t_{R}=7.84 min.

### N-(2-Cyclopropylpropan-2-yl)-2-(4-fluorophenyl)-4-methylpyrrolo[1,2-a][1,3,5]triazine-8-carboxamide

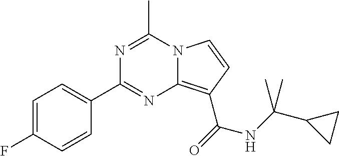 [SMILES: Cc1nc(-c2ccc(F)cc2)nc2c(C(=O)NC(C)(C)C3CC3)ccn12]

To a stirred solution of 4-fluorobenzonitrile (5.0 g, 41 mmol) in dioxane (4M HCl solution) was added EtOH (2.2 g, 49 mmol). The mixture was stirred at RT for 24 h, and concentrated in vacuo to afford ethyl 4-fluorobenzimidate hydrochloride (6.0 g) as a yellow solid. LC-MS m/z: 167.1 [M+H]⁺. t_{R}=0.98 min.

A mixture of ethyl 4-fluorobenzimidate hydrochloride (6.0 g, 36.0 mmol) and dimethylamine (2M in THF, 54 mL, 108 mmol) was stirred at RT for 24 h, and concentrated in vacuo. The residues was purified by silica gel chromatography (PE/EA; 1:1) to afford 4-fluoro-N,N-dimethylbenzimidamide (0.6 g, 10%) as a colorless oil. LC-MS m/z: 167.1 [M+H]⁺. t_{R}=0.98 min.

To a mixture of 4-fluoro-N,N-dimethylbenzimidamide (0.6 g, 3.6 mmol) and Et₃N (1.0 g, 10.8 mmol) in DCM (50 mL) was added acetyl chloride (340 mg, 4.5 mmol) at 0° C. Then the mixture was stirred at RT for 2 h, and concentrated in vacuo. The residue was purified by silica gel chromatography (PE/EA; 1:1) to afford N-((dimethylamino)(4-fluorophenyl)methylene)acetamide (0.3 g, 40%) as a colorless oil. LC-MS m/z: 209.1 [M+H]⁺. t_{R}=1.01 min.

Into the stirred solution of N-((dimethylamino)(4-fluorophenyl)methylene)acetamide (0.3 g, 1.4 mmol) and ethyl 2-amino-1H-pyrrole-3-carboxylate (0.22 g, 1.4 mmol) in toluene (10 mL) was added 4-methylbenzenesulfonic acid (17 mg, 0.1 mmol). The mixture was stirred at 100° C. for 24 h, and concentrated in vacuo. The residue was purified by silica gel chromatography (PE/EA; 2:1) to afford ethyl 2-(4-fluorophenyl)-4-methylpyrrolo[1,2-a][1,3,5]triazine-8-carboxylate (100 mg, 35%) as a yellow solid. LC-MS m/z: 300.1 [M+H]⁺. t_{R}=1.94 min.

Following general procedure B, ethyl 2-(4-fluorophenyl)-4-methylpyrrolo[1,2-a][1,3,5]triazine-8-carboxylate (0.1 g, 0.3 mmol) afforded 2-(4-fluorophenyl)-4-methylpyrrolo[1,2-a][1,3,5]triazine-8-carboxylic acid as a yellow solid (30 mg, 37%). LC-MS m/z: 272.1 [M+H]⁺. t_{R}=1.68 min.

Following general procedure A, 2-(4-fluorophenyl)-4-methylpyrrolo[1,2-a][1,3,5]triazine-8-carboxylic acid (22 mg, 0.08 mmol) and 2-cyclopropylpropan-2-amine afforded the title compound (2.9 mg, 10%) as a yellow solid. ¹H NMR (500 MHz, CDCl₃) δ 8.48 (dd, J=8.5 Hz, 6.0 Hz, 2H), 8.33 (brs, 1H), 7.56 (d, J=3.5 Hz, 1H), 7.18 (t, J=8.5 Hz, 2H), 7.16 (d, J=3.5 Hz, 1H), 2.86 (s, 3H), 1.48 (s, 6H), 1.48-1.43 (m, 1H), 0.56-0.53 (m, 4H). LC-MS m/z: 353.1 [M+H]⁺. HPLC: Purity (254 nm): 93%, t_{R}=9.76 min.

### N-(1-Cyclopropyl-2,2,2-trifluoroethyl)-2-(4-fluorophenyl)-4-methylpyrrolo[1,2-a][1,3,5]triazine-8-carboxamide

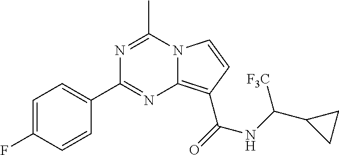 [SMILES: Cc1nc(-c2ccc(F)cc2)nc2c(C(=O)NC(C3CC3)C(F)(F)F)ccn12]

Following general procedure A, 2-(4-fluorophenyl)-4-methylpyrrolo[1,2-a][1,3,5]triazine-8-carboxylic acid (0.1 g, 0.3 mmol) and 1-cyclopropyl-2,2,2-trifluoroethanamine hydrochloride afforded the title compound (32 mg, 22%) as a white solid. ¹H NMR (400 MHz, DMSO-d₆) δ 8.61 (d, J=9.6 Hz, 1H), 8.43 (dd, J=8.4 Hz, 5.2 Hz, 2H), 7.73 (d, J=3.6 Hz, 1H), 7.44 (t, J=9.2 Hz, 2H), 7.39 (d, J=3.2 Hz, 1H), 4.48-4.42 (m, 1H), 2.91 (s, 3H), 1.36-1.30 (m, 1H), 0.72-0.56 (m, 3H), 0.41-0.37 (m, 1H). LC-MS m/z: 393.1 [M+H]⁺. HPLC: Purity (254 nm): >99%, t_{R}=9.58 min.

### N-(1-Cyclopropyl-2,2,2-trifluoroethyl)-2-(3-fluorophenyl)-4-methylpyrrolo[1,2-a][1,3,5]triazine-8-carboxamide

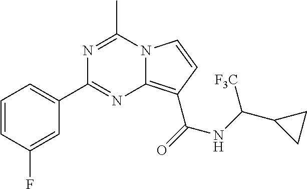 [SMILES: Cc1nc(-c2cccc(F)c2)nc2c(C(=O)NC(C3CC3)C(F)(F)F)ccn12]

To a solution of 3-fluorobenzonitrile (6.05 g, 50 mmol) in HCl-dioxane (4 M, 25 mL) was added EtOH (3.5 ml, 2.76 g, 60 mmol). The reaction mixture was stirred at 40° C. for 18 hours and stopped even the conversion was not complete. The reaction mixture was concentrated in vacuo, and the residue was triturated with MTBE once (20 mL) and filtered to afford ethyl 3-fluorobenzimidate hydrochloride (4.15 g, 41%) as a white solid.

A solution of ethyl 3-fluorobenzimidate hydrochloride (3.25 g, 19.5 mmol) in dimethylamine (2M in THF, 48.6 mL, 97.3 mmol) was stirred at 40° C. in a sealed tube overnight, and concentrated in vacuo to afford 3-fluoro-N,N-dimethylbenzimidamide (2.98 g, 92%) as a white solid.

A solution of 3-fluoro-N,N-dimethylbenzimidamide (2.98 g, 18.0 mmol) in DCM (25 ml) and pyridine (7.11 g, 90 mmol) was cooled to 0° C. with ice-water bath for 10 min. Then to the reaction mixture was added acetyl chloride (1.69 g, 21.6 mmol) slowly. After addition, the reaction mixture was warmed to RT, stirred for 2 h, washed with water (20 mL×2), dried over anhydrous Na₂SO₄, and filtered. The filtrate was concentrated in vacuo to afford N-((dimethylamino)(3-fluorophenyl)methylene)acetamide as brown oil (4.0 g, yield: 100%).

A mixture of N-((dimethylamino)(3-fluorophenyl)methylene)acetamide (2.08 g, 10 mmol) and ethyl 2-amino-1H-pyrrole-3-carboxylate (3.08 g, 20 mmol) in 20 mL of toluene was stirred for 5 minutes at RT. Then to the solution was added p-TSA (850 mg, 0.1 mmol). After addition, the reaction mixture was stirred at 90° C. for 8 h, and concentrated in vacuo. The residue was purified by silica gel column (0-40% of EA in PE) to afford ethyl 2-(3-fluorophenyl)-4-methylpyrrolo[1,2-a][1,3,5]triazine-8-carboxylate (130 mg, 4%) as an off-white solid.

Following general procedure B, ethyl 2-(3-fluorophenyl)-4-methylpyrrolo[1,2-a][1,3,5]triazine-8-carboxylate (65 mg, 0.22 mmol) afforded 2-(3-fluorophenyl)-4-methylpyrrolo[1,2-a][1,3,5]triazine-8-carboxylic acid (60 mg, 100%) as a brown solid.

Following general procedure A, 2-(3-fluorophenyl)-4-methylpyrrolo[1,2-a][1,3,5]triazine-8-carboxylic acid (30 mg, 0.102 mmol) and 1-cyclopropyl-2,2,2-trifluoroethanamine hydrochloride afforded the title compound (22.2 mg, 51%) as a yellow solid. ¹HNMR (500 MHz, MeOD-d₄) δ 8.31 (d, J=8.0 Hz, 1H), 8.17 (d, J=10.0 Hz, 1H), 7.62 (d, J=3.0 Hz, 1H), 7.58 (dd, J=14.0 Hz, 8.0 Hz, 1H), 7.49 (d, J=3.5 Hz, 1H), 7.32 (dd, J=8.0 Hz, 1.5 Hz, 1H), 4.53-4.46 (m, 1H), 2.98 (s, 3H), 1.37-1.30 (m, 1H), 0.82-0.76 (m, 1H), 0.72-0.64 (m, 1H), 0.61-0.51 (m, 2H). LC-MS m/z: 393.1 [M+H]⁺. HPLC Purity (214 nm): >99%; t_{R}=9.54 min.

### 2-(4-Fluoro-3-methoxyphenyl)-4-methyl-N-(1,1,1-trifluoropropan-2-yl)pyrrolo[1,2-a][1,3,5]triazine-8-carboxamide

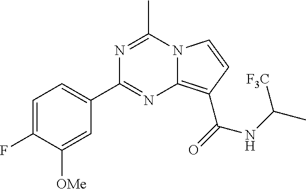 [SMILES: COc1cc(-c2nc(C)n3ccc(C(=O)NC(C)C(F)(F)F)c3n2)ccc1F]

To a solution of 4-fluoro-3-methoxybenzonitrile (10 g, 66.2 mmol) in HCl-dioxane (4 M, 50 mL) was added EtOH (3.66 g, 79.4 mmol). The reaction mixture was stirred at 40° C. for 18 hours and stopped even if the conversion was not complete. Then, the reaction mixture was concentrated in vacuo, and the resulting residue was triturated with MTBE once (20 mL) and filtered to afford ethyl 4-fluoro-3-methoxybenzimidate hydrochloride (13 g, 84%) as a white solid.

A solution of ethyl 4-fluoro-3-methoxybenzimidate hydrochloride (13 g, 55.8 mmol) in dimethylamine (2M in THF, 60 mL) was stirred at 40° C. in a sealed tube overnight, and then concentrated in vacuo to afford 4-fluoro-3-methoxy-N,N-dimethylbenzimidamide (11 g, 100%) as a white solid.

A solution of 4-fluoro-3-methoxy-N,N-dimethylbenzimidamide (11 g, 56.1 mmol) in DCM (50 mL) and pyridine (7.11 g, 280.5 mmol) was cooled to 0° C. for 10 min followed by the slow addition of acetyl chloride (5.25 g, 67.3 mmol). After complete addition, the reaction mixture was warmed to RT, stirred for 2 h, washed with water (50 mL×2), dried over anhydrous Na₂SO₄, and filtered. The filtrate was concentrated in vacuo to afford N-((dimethylamino)(4-fluoro-3-methoxyphenyl)methylene)acetamide (13.5 g, 100%) as a brown oil.

A mixture of N-((dimethylamino)(4-fluoro-3-methoxyphenyl)methylene)acetamide (500 mg, 2.1 mmol) and ethyl 2-amino-1H-pyrrole-3-carboxylate (324 mg, 2.1 mmol) in 20 mL of toluene was stirred for 5 minutes at RT followed by the addition of Et₃N (2.1 g, 21 mmol). The reaction mixture was then stirred at 110° C. for 12 h, concentrated in vacuo and the resulting residue purified by silica gel column chromatography (0-50% EA in PE) to afford ethyl 2-(4-fluoro-3-methoxyphenyl)-4-methylpyrrolo[1,2-a][1,3,5]triazine-8-carboxylate (120 mg, 17.4%) as an off-white solid.

Following general procedure B, ethyl 2-(4-fluoro-3-methoxyphenyl)-4-methylpyrrolo[1,2-a][1,3,5]triazine-8-carboxylate (120 mg, 0.36 mmol) afforded 2-(4-fluoro-3-methoxyphenyl)-4-methylpyrrolo[1,2-a][1,3,5]triazine-8-carboxylic acid sodium salt (150 mg, 100%) as a brown solid.

Following general procedure A, 2-(4-fluoro-3-methoxyphenyl)-4-methylpyrrolo[1,2-a][1,3,5]triazine-8-carboxylic acid sodium salt (100 mg, 0.31 mmol) and 1,1,1-trifluoropropan-2-amine hydrochloride afforded the title compound (33 mg, 27%) as a yellow solid. ¹HNMR (500 MHz, MeOD-d₄) δ 8.18-8.13 (m, 2H), 7.58 (d, J=3.0 Hz, 1H), 7.47 (d, J=3.0 Hz, 1H), 7.28 (t, J=8.5 Hz, 1H), 5.05-5.00 (m, 1H), 4.02 (s, 3H), 2.96 (s, 3H), 1.55 (d, J=6.5 Hz, 2H). LC-MS m/z: 396.8 [M+H]⁺. HPLC Purity (214 nm): 98%; t_{R}=9.14 min.

### N-(4-Ethynylphenyl)-2,4-dimethylquinazoline-8-carboxamide

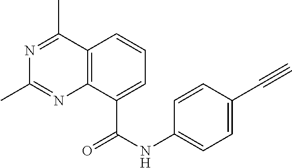 [SMILES: C#Cc1ccc(NC(=O)c2cccc3c(C)nc(C)nc23)cc1]

Following general procedure A, 2,4-dimethylquinazoline-8-carboxylic acid (50 mg, 0.25 mmol) and 4-ethynylaniline afforded the title compound (27.2 mg, 36%) as a yellow solid. ¹H NMR (500 MHz, DMSO-d₆): δ 13.25 (s, 1H), 8.75 (d, J=7.0 Hz, 1H), 8.54 (d, J=9.0 Hz, 1H), 7.88-7.83 (m, 3H), 7.53 (d, J=8.5 Hz, 2H), 4.16 (s, 1H), 2.98 (s, 3H), 2.90 (s, 3H). LC-MS m/z: 302.2 [M+H]⁺. HPLC Purity (214 nm): >99%; t_{R}=10.90 min.

### N-(2,3-Dihydro-1H-inden-5-yl)-2,4-dimethylquinazoline-8-carboxamide

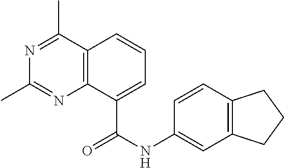 [SMILES: Cc1nc(C)c2cccc(C(=O)Nc3ccc4c(c3)CCC4)c2n1]

Following general procedure A, 2,4-dimethylquinazoline-8-carboxylic acid (50 mg, 0.25 mmol) and 2,3-dihydro-1H-inden-5-amine afforded the title compound (65 mg, 83%) as a yellow solid. ¹H NMR (500 MHz, MeOD-d₄): δ 8.89 (d, J=7.5 Hz, 1H), 8.48 (d, J=8.0 Hz, 1H), 7.82 (t, J=8.0 Hz, 1H), 7.71 (s, 1H), 7.48 (d, J=7.5 Hz, 1H), 7.24 (d, J=7.5 Hz, 1H), 3.02 (s, 3H), 2.95 (s, 3H), 3.02-2.90 (m, 4H), 2.14 (m, J=7.5 Hz, 2H). LC-MS m/z: 318.2 [M+H]⁺. HPLC Purity (214 nm): >99%; t_{R}=11.46 min.

### 2,4-Dimethyl-N-(1,2,3,4-tetrahydronaphthalen-1-yl)quinazoline-8-carboxamide

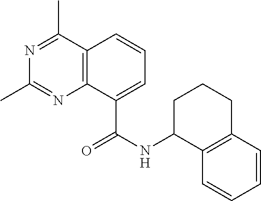 [SMILES: Cc1nc(C)c2cccc(C(=O)NC3CCCc4ccccc43)c2n1]

Following general procedure A, 2,4-dimethylquinazoline-8-carboxylic acid (50 mg, 0.25 mmol) and 1,2,3,4-tetrahydronaphthalen-1-amine afforded the title compound (74 mg, 90%) as a yellow oil. ¹H NMR (500 MHz, MeOD-d₄): δ 8.84 (d, J=7.0 Hz, 1H), 8.45 (d, J=8.0 Hz, 1H), 7.80 (t, J=7.0 Hz, 1H), 7.44 (d, J=7.0 Hz, 1H), 7.23-7.18 (m, 3H), 5.39 (t, J=7.5 Hz, 1H), 2.98 (s, 3H), 2.96-2.88 (m, 2H), 2.59 (s, 3H), 2.25-2.20 (m, 1H), 2.10-2.02 (m, 2H), 2.00-1.96 (m, 1H). LC-MS m/z: 332.3 [M+H]⁺. HPLC Purity (214 nm): 99%; t_{R}=10.89 min.

### 2,4-Dimethyl-N-(1-phenylpropyl)quinazoline-8-carboxamide

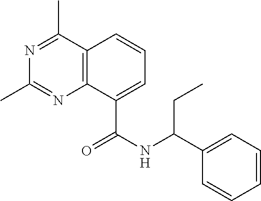 [SMILES: CCC(NC(=O)c1cccc2c(C)nc(C)nc12)c1ccccc1]

Following general procedure A, 2,4-dimethylquinazoline-8-carboxylic acid (50 mg, 0.25 mmol) and 1-phenylpropan-1-amine afforded the title compound (69.2 mg, 87%) as a white solid. ¹H NMR (500 MHz, MeOD-d₄): δ 8.79 (d, J=7.5 Hz, 1H), 8.48 (dd, J=8.5 Hz, 1.5 Hz, 1H), 7.79 (d, J=8.0 Hz, 1H), 7.48 (d, J=8.0 Hz, 2H), 7.40 (t, J=8.0 Hz, 2H), 7.30 (d, J=8.0 Hz, 1H), 5.14 (t, J=7.5 Hz, 1H), 3.02 (s, 3H), 2.88 (s, 3H), 2.11-2.00 (m, 2H), 1.04 (t, J=8.0 Hz, 3H). LC-MS m/z: 320.3 [M+H]⁺. HPLC Purity (214 nm): >99%; t_{R}=10.68 min.

### 2,4-Dimethyl-N-((1R,4R)-4-(pentyloxy)cyclohexyl)quinazoline-8-carboxamide

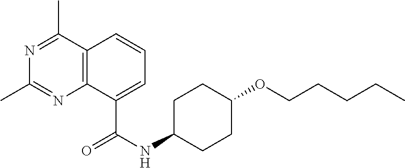 [SMILES: CCCCCO[C@H]1CC[C@H](NC(=O)c2cccc3c(C)nc(C)nc23)CC1]

Following general procedure A, 2,4-dimethylquinazoline-8-carboxylic acid (50 mg, 0.25 mmol) and (1R,4R)-4-(pentyloxy)cyclohexanamine afforded the title compound (74 mg, 81%) as a colorless oil. ¹H NMR (500 MHz, MeOD-d₄): δ 10.90 (d, J=9.5 Hz, 1H), 8.68 (dd, J=7.5 Hz, 1.5 Hz, 1H), 8.45 (dd, J=7.5 Hz, 1.5 Hz, 1H), 7.78 (t, J=7.5 Hz, 1H), 3.92-3.88 (m, 1H), 3.41 (t, J=6.5 Hz, 2H), 2.94 (s, 3H), 2.79 (s, 3H), 0.08-2.02 (m, 2H), 2.00-1.94 (m, 2H), 1.49-1.23 (m, 11H), 0.88 (t, J=7.0 Hz, 3H). LC-MS m/z: 370.3 [M+H]⁺. HPLC Purity (214 nm): 99%; t_{R}=11.93 min.

### Example 2—Preparation of Additional Pyrrolo[1,2-a]triazine Compounds

Additional pyrrolo[1,2-a]triazine compounds were prepared based on the general procedure described below.

### Part I—General Procedures

General Procedure A: Preparation of Amide by Coupling of a Carboxylic Acid Compound with an Amine Compound

To a stirred solution of carboxylic acid compound (1.0 equivalent), HATU (1.5 equivalents), and DIPEA (3.75 equivalents) in DCM or DMF (˜4 mL/0.2 mmol) was added the amine compound (1.25-2.0 equivalents). The reaction mixture was stirred at room temperature for 4-16 hours, and then washed with saturated aqueous NaHCO₃ solution (5 mL/0.2 mmol), aqueous citric acid solution (5 mL/0.2 mmol) and brine (5 mL/0.2 mmol). The combined extracts were dried over anhydrous Na₂SO₄, filtered and concentrated in vacuo. The resulting crude material was purified by silica gel column chromatography or preparatory HPLC to give the amide compound.

### General Procedure B: Conversion of Carboxylic Ester Compound to Carboxylic Acid Compound

To a solution of carboxylic ester (1.0 equivalent) in EtOH (5.0 mL/1.0 mmol) and water (0-3.0 mL/1.0 mmol) was added NaOH (2.0-5.0 equivalents) and the mixture was heated at 80° C. for 2 h and then concentrated. To the concentrate, 6N HCl solution was added to adjust the pH to 5-6 and the mixture was stirred for 10 minutes and then filtered. The resulting solid was collected and dried to give the carboxylic acid.

### Part II—Preparation of 2-(3-Cyanophenyl)-N-(1-cyclopropyl-2,2,2-trifluoroethyl)-4-methylpyrrolo[1,2-a][1,3,5]triazine-8-carboxamide & 2-(3-carbamoylphenyl)-N-(1-cyclopropyl-2,2,2-trifluoroethyl)-4-methylpyrrolo[1,2-a][1,3,5]triazine-8-carboxamide

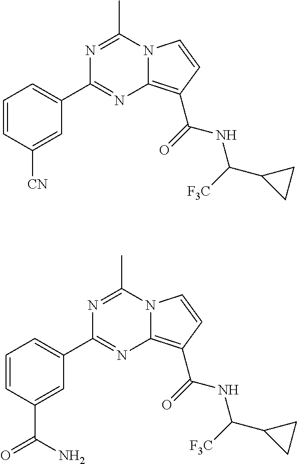 [SMILES: Cc1nc(-c2cccc(C#N)c2)nc2c(C(=O)NC(C3CC3)C(F)(F)F)ccn12 ; Cc1nc(-c2cccc(C(N)=O)c2)nc2c(C(=O)NC(C3CC3)C(F)(F)F)ccn12]

To a solution of 3-bromobenzonitrile (5.0 g, 27.6 mmol) in HCl-dioxane (4 M, 10 mL) was added EtOH (1.52 g, 33.12 mmol) and the reaction mixture was at stirred at RT for 16 h and concentrated in vacuo. The residue was triturated with MTBE (20 mL) and filtered to afford ethyl 3-bromobenzimidate hydrochloride (4.0 g, 59%) as a white solid. LC-MS m/z: 229 [M+H]⁺.

A solution of ethyl 3-bromobenzimidate hydrochloride (4.0 g, 16.4 mmol) in Me₂NH (2M in THF, 40 mL) was stirred at RT for 16 h, and concentrated in vacuo to afford 3-bromo-N,N-dimethylbenzimidamide (3.0 g, 80%) as light yellow oil. LC-MS m/z: 227.1 [M+H]⁺.

A solution of 3-bromo-N,N-dimethylbenzimidamide (3.0 g, 13.2 mmol) and pyridine (6.67 g, 66.0 mmol) in DCM (100 mL) was cooled to 0° C. and then acetyl chloride (1.25 g, 15.8 mmol) was added slowly to the reaction mixture. The reaction mixture was warmed to RT, stirred for 16 h, washed with H₂O (50 mL×2), dried over anhydrous Na₂SO₄, and filtered. The filtrate was concentrated in vacuo to afford N-((3-bromophenyl)(dimethylamino)methylene) acetamide (3.0 g, 84%) as a light yellow oil. LC-MS m/z: 269.1 [M+H]⁺.

A mixture of N-((3-bromophenyl)(dimethylamino)methylene)acetamide (3.0 g, 11.1 mmol) and ethyl 2-amino-1H-pyrrole-3-carboxylate (1.72 g, 11.1 mmol) in 50 mL of toluene was stirred for 5 min at RT and then Et₃N (2.24 g, 22.2 mmol) was added to the reaction mixture. The reaction mixture was stirred at 110° C. for 12 h, concentrated in vacuo and the residue was purified by silica gel column chromatography (PE/EA=4/1) to afford ethyl 2-(3-bromophenyl)-4-methylpyrrolo[1,2-a][1,3,5]triazine-8-carboxylate (360 mg, 90%) as a grey solid. LC-MS m/z: 360.1 [M+H]⁺.

A mixture of ethyl 2-(3-bromophenyl)-4-methylpyrrolo[1,2-a][1,3,5]triazine-8-carboxylate (280 mg, 0.78 mmol), Pd(P^{t}Bu₃)₂Cl₂ (40 mg, 0.078 mmol) and Zn(CN)₂ (182 mg, 1.56 mmol) in DMF (10 mL) was stirred at 100° C. under N₂ for 12 h, poured into H₂O (20 mL), and extracted with EA (30 mL×3). The combined organic phases were dried over anhydrous Na₂SO₄, filtered and concentrated in vacuo. The residue was purified by silica gel column chromatography (PE/EA: 4/1) to afford ethyl 2-(3-cyanophenyl)-4-methylpyrrolo[1,2-a][1,3,5]triazine-8-carboxylate (200 mg, 84%) as a grey solid. LC-MS m/z: 307.1 [M+H]⁺.

Following general procedure B, ethyl 2-(3-cyanophenyl)-4-methylpyrrolo[1,2-a][1,3,5]triazine-8-carboxylate (200 mg, 0.65 mmol) afforded a mixture of 2-(3-cyanophenyl)-4-methylpyrrolo[1,2-a][1,3,5]triazine-8-carboxylic acid and 2-(3-carbamoylphenyl)-4-methylpyrrolo[1,2-a][1,3,5]triazine-8-carboxylic acid (280 mg, crude) as brown solids which were used in the next step without purification. LC-MS m/z: 279.1 [M+H]⁺.

Following general procedure A, a mixture of 2-(3-cyanophenyl)-4-methylpyrrolo[1,2-a][1,3,5]triazine-8-carboxylic acid and 2-(3-carbamoylphenyl)-4-methylpyrrolo[1,2-a][1,3,5]triazine-8-carboxylic acid (110 mg, 0.39 mmol) and 1-cyclopropyl-2,2,2-trifluoroethanamine hydrochloride afforded 2-(3-cyanophenyl)-N-(1-cyclopropyl-2,2,2-trifluoroethyl)-4-methylpyrrolo[1,2-a][1,3,5]triazine-8-carboxamide (40 mg, 22%) and 2-(3-carbamoylphenyl)-N-(1-cyclopropyl-2,2,2-trifluoroethyl)-4-methylpyrrolo[1,2-a][1,3,5]triazine-8-carboxamide (28 mg, 15%) as yellow solids.

2-(3-Cyanophenyl)-N-(1-cyclopropyl-2,2,2-trifluoroethyl)-4-methylpyrrolo[1,2-a][1,3,5]triazine-8-carboxamide: ¹H NMR (400 MHz, DMSO-d₆): δ 8.68 (s, 1H), 8.64 (d, J=8.0 Hz, 1H), 8.56 (d, J=9.2 Hz, 1H), 8.05 (d, J=7.6 Hz, 1H), 7.82 (t, J=8.0 Hz, 1H), 7.79 (d, J=3.2 Hz, 1H), 7.45 (d, J=3.6 Hz, 1H), 4.51-4.45 (m, 1H), 2.94 (s, 3H), 1.37-1.32 (m, 1H), 0.73-0.68 (m, 1H), 0.63-0.58 (m, 2H), 0.44-0.40 (m, 1H). LC-MS m/z: 400.1 [M+H]⁺. HPLC: Purity (214 nm): >99%; t_{R}=8.92 min.

2-(3-Carbamoylphenyl)-N-(1-cyclopropyl-2,2,2-trifluoroethyl)-4-methylpyrrolo[1,2-a][1,3,5]triazine-8-carboxamide: ¹H NMR (500 MHz, MeOD-d₄): δ 9.03 (s, 1H), 8.63 (d, J=8.0 Hz, 1H), 8.03 (d, J=8.0 Hz, 1H), 7.65 (t, J=8.0 Hz, 1H), 7.61 (d, J=3.0 Hz, 1H), 7.49 (d, J=3.5 Hz, 1H), 4.41-4.38 (m, 1H), 2.99 (s, 3H), 1.45-1.42 (m, 1H), 0.83-0.80 (m, 1H), 0.73-0.70 (m, 1H), 0.63-0.60 (m, 1H), 0.54-0.51 (m, 1H). LC-MS m/z: 418.1 [M+H]⁺. HPLC: Purity (214 nm): 97%; t_{R}=7.26 min.

### Example 3—Biological Activity Evaluation

The ability of exemplary compounds to activate glucocerebrosidase (Gcase) was measured. Experimental procedures and results are provided below.

### Part I: Assay Procedure

A 484 μL aliquot of a 1.0 mg/mL solution of phosphatidylserine (PS) (Sigma P7769) in chloroform was evaporated under a stream of nitrogen for 1 hour. The lipid film was dissolved over 4 minutes of vigorous vortexing in 40 mL of 176 mM K₂HPO₄/50 mM citric acid (pH 4.7) containing 7.5 μL of triton X-100, resulting in a mixed micellar preparation with a composition of 0.32 mM triton and 0.37 mol % PS. 4-Methylumbelliferyl-beta-D-glucopyranoside (ACROS-337025000) was dissolved in the micellar solution to a final concentration of 2 mM for use as the reaction substrate.

Test compounds were diluted to the desired concentrations with dimethylsulfoxide (DMSO) from 10 mM stocks, and 0.41 μL of the DMSO compound mixture was added to 100 μL of micellar solution containing 10 nM GCase and 100 nM saposin C (Enzo ALX-201-262-C050). Pre-incubation was allowed to occur for 30 minutes at room temperature, after which the reaction was initiated by combining 25 μL of substrate solution with 25 μL of compound/GCase/saposin mixture. The reaction proceeded for 15 minutes at room temperature and was stopped by adding 150 μL of 1M glycine, pH 12.5. The endpoint of the reaction was monitored by measuring fluorescence intensity (excitation: 365 nm; emission: 440 nm) on a SpectraMax i3 instrument (Molecular Devices). Test compounds were screened at 1.0 and 0.1 M final concentration, and subsequent 8-point dose response curves were obtained using 3-fold dilutions from a maximum final concentration of 5 μM.

### Part II: Results

Gcase activation values for tested compounds are provided in Tables 4 and 5 below, along with cLogP, PSA, and compound solubility in water. The symbol “+” indicates less than 5% Gcase activation; the symbol “++” indicates Gcase activation in the range of 5% up to 20%; and the symbol “+++” indicates Gcase activation greater than 20%. The symbol “N/A” indicates that no data is available.

TABLE 4

| | | | Compound | | |
|---|---|---|---|---|---|
| | | | Solubility in | Percent Gcase Activation | |
| | | | Water | 1 μM Test | 0.1 μM Test |
| Compound Structure | cLogP | PSA | (μg/mL) | Compound | Compound |
| 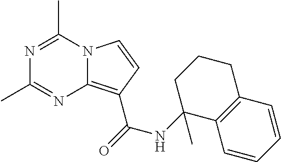 [SMILES: Cc1nc(C)n2ccc(C(=O)NC3(C)CCCc4ccccc43)c2n1] | 4.3 | 57.1 | 5.1 | +++ | ++ |
| 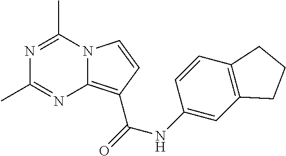 [SMILES: Cc1nc(C)n2ccc(C(=O)Nc3ccc4c(c3)CCC4)c2n1] | 3.6 | 57.1 | 1.2 | +++ | + |
| 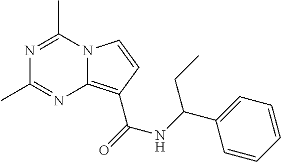 [SMILES: CCC(NC(=O)c1ccn2c(C)nc(C)nc12)c1ccccc1] | 3.6 | 57.1 | 17.39 | +++ | ++ |
| 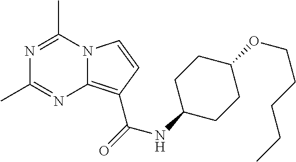 [SMILES: CCCCCO[C@H]1CC[C@H](NC(=O)c2ccn3c(C)nc(C)nc23)CC1] | 3.4 | 66.3 | 12.54 | +++ | ++ |
| 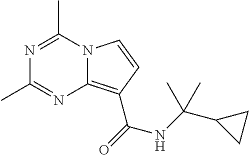 [SMILES: Cc1nc(C)n2ccc(C(=O)NC(C)(C)C3CC3)c2n1] | 2.5 | 57.1 | 38.4 | ++ | + |
| 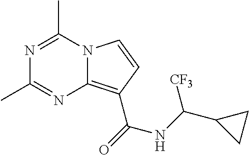 [SMILES: Cc1nc(C)n2ccc(C(=O)NC(C3CC3)C(F)(F)F)c2n1] | 2.4 | 57.1 | 28.2 | +++ | ++ |
| 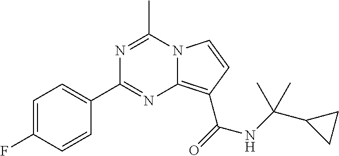 [SMILES: Cc1nc(-c2ccc(F)cc2)nc2c(C(=O)NC(C)(C)C3CC3)ccn12] | 4.2 | 57.1 | 0.5 | +++ | + |
| 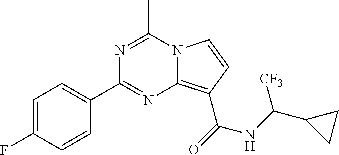 [SMILES: Cc1nc(-c2ccc(F)cc2)nc2c(C(=O)NC(C3CC3)C(F)(F)F)ccn12] | 4.1 | 57.1 | 0.1 | +++ | +++ |
| 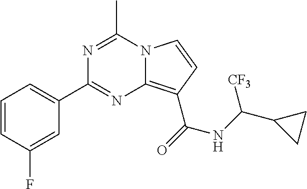 [SMILES: Cc1nc(-c2cccc(F)c2)nc2c(C(=O)NC(C3CC3)C(F)(F)F)ccn12] | 4.1 | 57.1 | 0.2 | +++ | +++ |
| 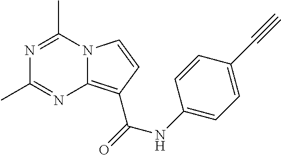 [SMILES: C#Cc1ccc(NC(=O)c2ccn3c(C)nc(C)nc23)cc1] | 2.9 | 57.1 | 1.8 | +++ | + |
| 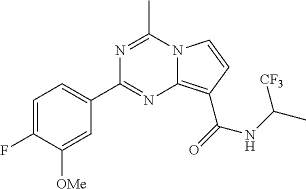 [SMILES: COc1cc(-c2nc(C)n3ccc(C(=O)NC(C)C(F)(F)F)c3n2)ccc1F] | 3.5 | 66.3 | 1.0 | +++ | +++ |
| 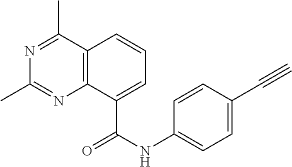 [SMILES: C#Cc1ccc(NC(=O)c2cccc3c(C)nc(C)nc23)cc1] | 3.5 | 53.8 | 0.7 | + | + |
| 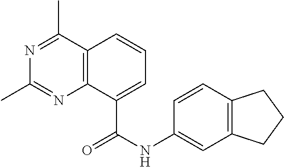 [SMILES: Cc1nc(C)c2cccc(C(=O)Nc3ccc4c(c3)CCC4)c2n1] | 4.3 | 53.8 | 0.5 | ++ | + |
| 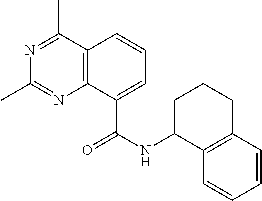 [SMILES: Cc1nc(C)c2cccc(C(=O)NC3CCCc4ccccc43)c2n1] | 4.4 | 53.8 | 5.0 | ++ | + |
| 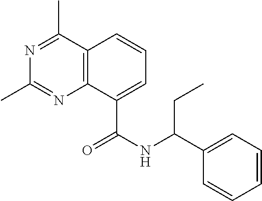 [SMILES: CCC(NC(=O)c1cccc2c(C)nc(C)nc12)c1ccccc1] | 4.3 | 53.8 | 8.7 | ++ | + |
| 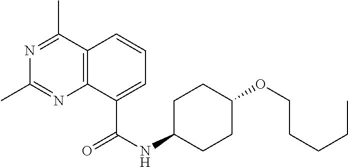 [SMILES: CCCCCO[C@H]1CC[C@H](NC(=O)c2cccc3c(C)nc(C)nc23)CC1] | 4.1 | 63.1 | 7.6 | ++ | + |

TABLE 5

| | | | Compound | | |
|---|---|---|---|---|---|
| | | | Solubility in | Percent Gcase Activation | |
| | | | Water | 1 μM Test | 0.1 μM Test |
| Compound Structure | cLogP | PSA | (μg/mL) | Compound | Compound |
| 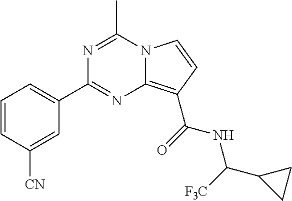 [SMILES: Cc1nc(-c2cccc(C#N)c2)nc2c(C(=O)NC(C3CC3)C(F)(F)F)ccn12] | 3.4 | 80.9 | 0.6 | +++ | +++ |
| 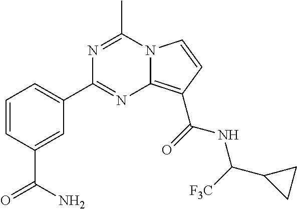 [SMILES: Cc1nc(-c2cccc(C(N)=O)c2)nc2c(C(=O)NC(C3CC3)C(F)(F)F)ccn12] | 2.6 | 100.2 | 1.9 | +++ | +++ |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound of Formula I: 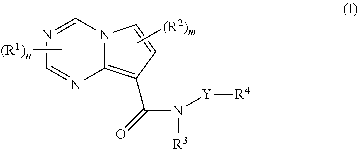
or a pharmaceutically acceptable salt thereof, wherein:
R¹ and R² each represent independently for each occurrence hydrogen, C₁-C₆ alkyl, halogen, C₁-C₆ haloalkyl, C₃-C₆ cycloalkyl, C₁-C₆ alkoxy, —(C₁-C₄ alkylene)-O—(C₁-C₆ alkyl), —(C₁-C₄ alkylene)-O—(C₃-C₆ cycloalkyl), 3-6 membered heterocyclyl, 6-membered aryl, cyano, or —N(R⁵)₂;
R³ is hydrogen, C₁-C₆ alkyl, or C₃-C₆ cycloalkyl;
R⁴ is one of the following:
C₃-C₈ cycloalkyl, 3-8 membered heterocycloalkyl, 9-13 membered spiroheterocycloalkyl, —(C₂-C₆ alkylene)-O-phenyl, phenyl, heteroaryl, a partially unsaturated 9-10 membered bicyclic carbocyclyl, or a partially unsaturated 8-10 membered bicyclic heterocyclyl; each of which is optionally substituted by 1, 2, or 3 substituents independently selected from the group consisting of C₁-C₈ alkyl, halogen, C₁-C₆ haloalkyl, C₃-C₆ cycloalkyl, —(C₁-C₆ alkylene,)—(C₃-C₆ cycloalkyl), hydroxyl, C₁-C₆ alkoxy, —O—(C₃-C₆ cycloalkyl), C₂-C₄ alkynyl, —(C₂-C₄ alkynyl)-C₁-C₆ alkoxy, aryl, —O-aryl, heteroaryl, saturated 3-8 membered heterocyclyl, amino, and —CO₂R⁵; or
C₁-C₆ alkyl;
R⁵ represents independently for each occurrence hydrogen, C₁-C₆ alkyl, or C₃-C₆ cycloalkyl;
Y is a bond, —C(O)—, C₁-C₆ alkylene, C₁-C₆ haloalkylene, or C₃-C₆ cycloalkylene; and
n and m are independently 1 or 2.

2. The compound of claim 1, wherein R¹ is C₁-C₃ alkyl.

3. The compound of claim 1, wherein R² is hydrogen.

4. The compound of claim 1, wherein R³ is hydrogen.

5. The compound of claim 1, wherein Y is a bond.

6. The compound of claim 1, wherein Y is C₁-C₆ alkylene.

7. The compound of claim 1, wherein R⁴ is C₃-C₈ cycloalkyl optionally substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, and —(C₂-C₄ alkynyl)-C₁-C₆ alkoxy.

8. The compound of claim 1, wherein R⁴ is a partially unsaturated 9-10 membered bicyclic carbocyclyl optionally substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, —(C₂-C₄ alkynyl)-C₁-C₆ alkoxy, aryl, heteroaryl, and saturated 3-8 membered heterocyclyl.

9. The compound of claim 1, wherein R⁴ is a partially unsaturated 9-10 membered bicyclic carbocyclyl optionally substituted by C₁-C₆ alkyl.

10. The compound of claim 1, wherein R⁴ is phenyl substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, —(C₂-C₄ alkynyl)-C₁-C₆ alkoxy, 5-membered heteroaryl, and saturated 3-8 membered heterocyclyl.

11. The compound of claim 1, wherein R⁴ is phenyl substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, and —(C₂-C₄ alkynyl)-C₁-C₆ alkoxy.

12. The compound of claim 1, wherein n is 2.

13. The compound of claim 1, wherein m is 1.

14. The compound of claim 1, wherein the compound is represented by Formula I-A: 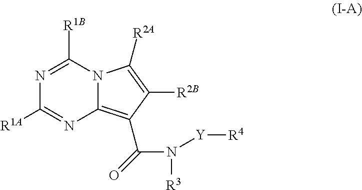
or a pharmaceutically acceptable salt thereof, wherein:
R^{1A}, R^{1B}, R^{2A} and, R^{2B} are independently hydrogen, C₁-C₃ alkyl, or cyclopropyl;
R³ is hydrogen;
R⁴ is C₃-C₈ cycloalkyl, 3-8 membered heterocycloalkyl, 9-13 membered spiroheterocycloalkyl, —(C₂-C₆ alkylene)-O-phenyl, phenyl, heteroaryl, a partially unsaturated 9-10 membered bicyclic carbocyclyl, or a partially unsaturated 8-10 membered bicyclic heterocyclyl; each of which is optionally substituted by 1, 2, or 3 substituents independently selected from the group consisting of C₁-C₈ alkyl, halogen, C₁-C₆ haloalkyl, C₃-C₆ cycloalkyl, —(C₁-C₆ alkylene,)—(C₃-C₆ cycloalkyl), hydroxyl, C₁-C₆ alkoxy, C₂-C₄ alkynyl, and —(C₂-C₄ alkynyl)-C₁-C₆ alkoxy; and
Y is a bond or C₁-C₆ alkylene.

15. The compound of claim 14, wherein R^{1A} and R^{1B} are methyl.

16. The compound of claim 14, wherein R^{2A} and R^{2B}are hydrogen.

17. The compound of claim 14, wherein Y is a bond.

18. The compound of claim 14, wherein Y is C₁-C₆ alkylene.

19. The compound of claim 14, wherein R⁴ is C₃-C₈ cycloalkyl optionally substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, and —(C₂-C₄ alkynyl)-C₁-C₆ alkoxy.

20. The compound of claim 14, wherein R⁴ is C₄-C₆ cycloalkyl optionally substituted by 1 or 2 substituents independently selected from the group consisting of C₁-C₆ alkyl, C₁-C₆ haloalkyl, C₃-C₆ cycloalkyl, and C₁-C₆ alkoxy.

21. The compound of claim 14, wherein R⁴ is a partially unsaturated 9-10 membered bicyclic carbocyclyl optionally substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, and —(C₂-C₄ alkynyl)-C₁-C₆ alkoxy.

22. The compound of claim 14, wherein R⁴ is a partially unsaturated 9-10 membered bicyclic carbocyclyl optionally substituted by C₁-C₆ alkyl.

23. The compound of claim 14, wherein R⁴ is phenyl substituted by 1, 2, or 3 substituents independently selected from the group consisting of C₁-C₆ alkyl, halogen, C₁-C₆ haloalkyl, C₃-C₆ cycloalkyl, hydroxyl, C₁-C₆ alkoxy, C₂-C₄ alkynyl, and —(C₂-C₄ alkynyl)-C₁-C₆ alkoxy.

24. The compound of claim 14, wherein R⁴ is phenyl substituted by 1, 2, or 3 substituents independently selected from the group consisting of C₁-C₆ alkyl, halogen, C₁-C₆ haloalkyl, C₃-C₆ cycloalkyl, C₁-C₆ alkoxy, and C₂-C₄ alkynyl.

25-48. (canceled)

49. A compound of claim 1 selected from the group consisting of: 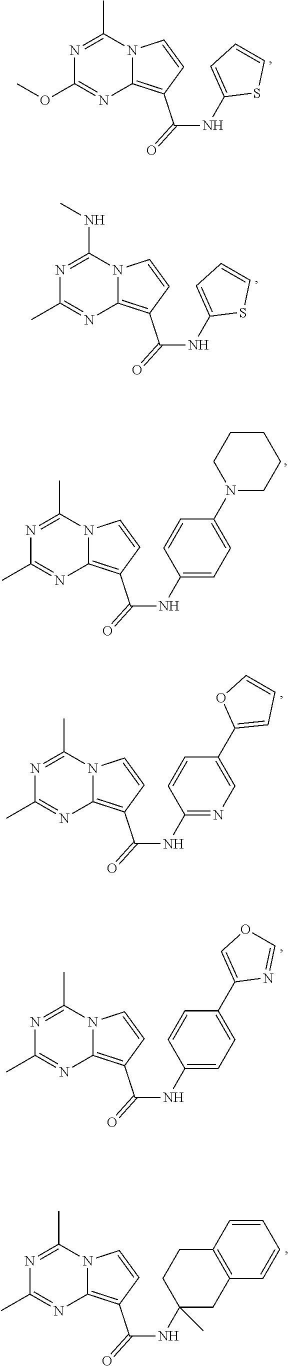 [SMILES: COc1nc(C)n2ccc(C(=O)Nc3cccs3)c2n1 ; CNc1nc(C)nc2c(C(=O)Nc3cccs3)ccn12 ; Cc1nc(C)n2ccc(C(=O)Nc3ccc(N4CCCCC4)cc3)c2n1 ; Cc1nc(C)n2ccc(C(=O)Nc3ccc(-c4ccco4)cn3)c2n1 ; Cc1nc(C)n2ccc(C(=O)Nc3ccc(-c4cocn4)cc3)c2n1 ; Cc1nc(C)n2ccc(C(=O)NC3(C)CCc4ccccc4C3)c2n1] 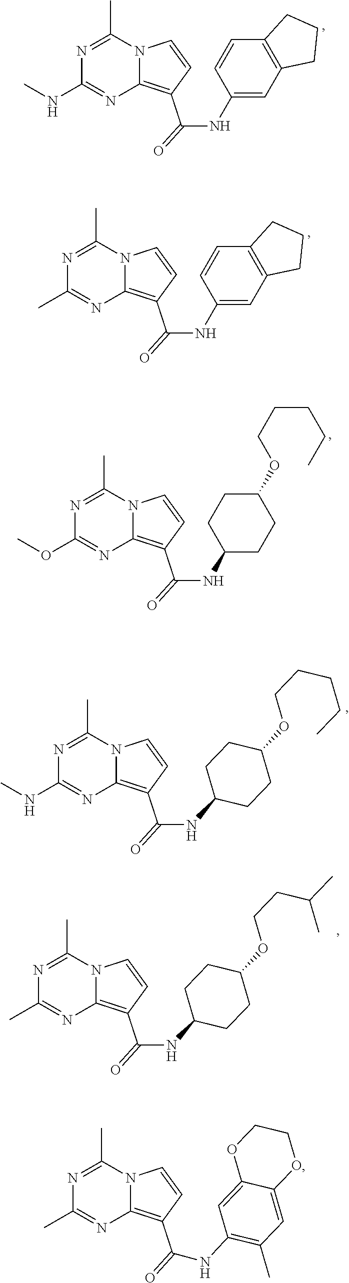 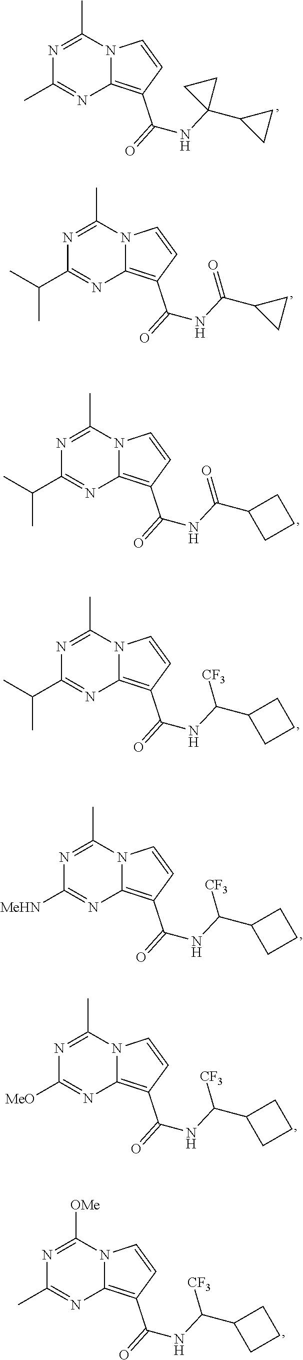 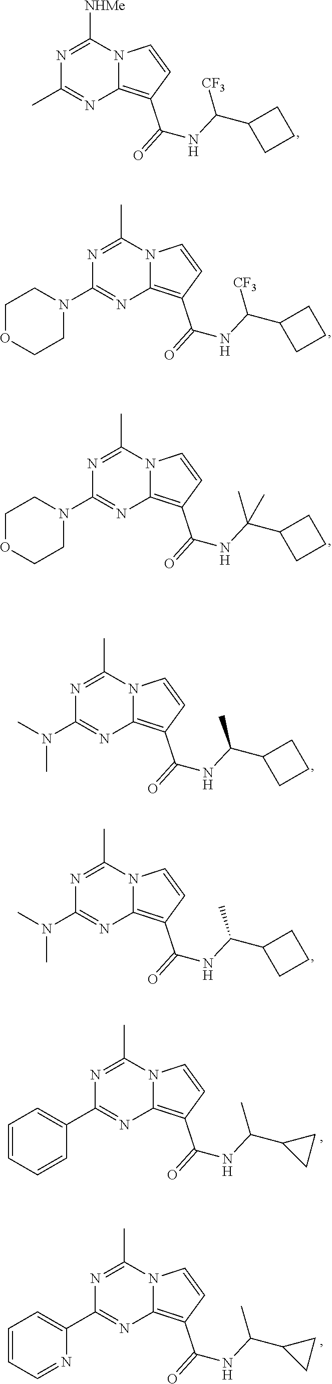 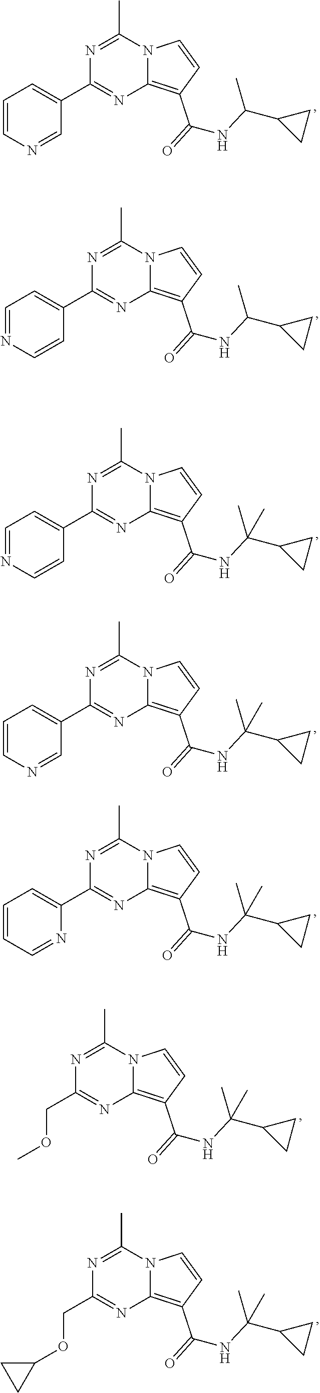 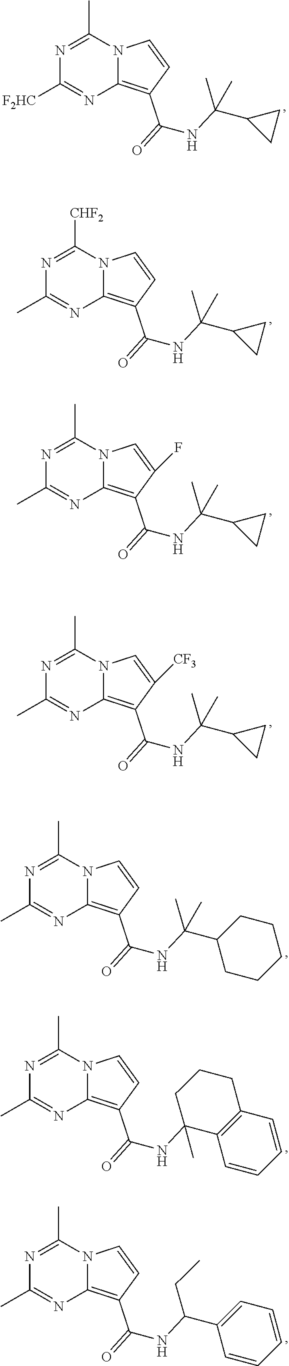 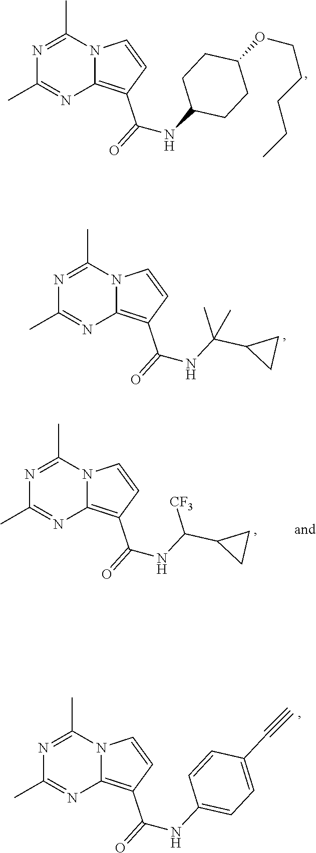
or a pharmaceutically acceptable salt thereof.

50. (canceled)

51. A pharmaceutical composition, comprising a compound of claim 1 and a pharmaceutically acceptable carrier.

52. (canceled)

53. A method of treating a disorder selected from the group consisting of Gaucher disease, Parkinson's disease, Lewy body disease, dementia, multiple system atrophy, epilepsy, bipolar disorder, schizophrenia, an anxiety disorder, major depression, polycystic kidney disease, type 2 diabetes, open angle glaucoma, multiple sclerosis, endometriosis, and multiple myeloma, comprising administering to a patient in need thereof a therapeutically effective amount of a compound of claim 1 to treat the disorder.

54-59. (canceled)
